SEQUENCE LISTING (2 sequences)

SEQ ID NO: 1 - protein, 547 aa, Pseudomonas stutzeri
MSHILRAAVLAAMLLPLPSMADQAGKSPNAVRYHGGDEIILQGFHWNVVREAPNDWYNILRQQAATIAADGFSAIWMPVPWRDFSSWSDGSKSGGGEGYFWHDFNKNGRYGSDAQLRQAASALGGAGVKVLYDVVPNHMNRGYPDKEINLPAGQGFWRNDCADPGNYPNDCDDGDRFIGGDADLNTGHPQVYGMFRDEFTNLRSQYGAGGFRFDFVRGYAPERVNSWMTDSADNSFCVGELWKGPSEYPNWDWRNTASWQQIIKDWSDRAKCPVFDFALKERMQNARSPTGSTPERQSRPAWREVAVTFVDNHDTGYSPGQNGGQHHWALQDGLIRQAYAYILTSPGTPVVYWSHMYDWGYGDFIRQLIQVRRAAGVRADSAISFHSGYSGLVATVSGSQQTLVVALNSDLGNPGQVASGSFSEAVNASNGQVRVWRSGTGSGGGEPGALVSVSFRCDNGATQMGDSVYAVGNVSQLGNWSPAAALRLTDTSGYPTWKGSIALPAGQNEEWKCLIRNEANATQVRQWQGGANNSLTPSEGATTVGRL

SEQ ID NO: 2 - DNA, 1644 bp, Pseudomonas stutzeri
atgagccacatcctgcgagccgccgtattggcggcgatgctgttgccgttgccgtccatggccgatcaggccggcaagagccccaacgctgtgcgctaccacggcggcgacgaaatcattctccagggctttcactggaacgtcgtccgcgaagcgcccaacgactggtacaacatcctgcgccagcaggccgcgaccatcgccgccgacggcttctcggcgatctggatgccggtgccctggcgcgacttctccagctggagcgacggcagcaagtccggcggcggtgaaggctacttctggcacgacttcaacaagaacggccgctatggcagtgacgcccagctgcgtcaggccgccagcgcgctcggtggcgccggcgtgaaagtgctttacgacgtggtgcccaaccacatgaaccgtggctatccggacaaggagatcaacctcccggccggccagggcttctggcgcaacgactgcgccgacccgggcaactaccccaatgattgcgacgacggcgaccgcttcatcggcggcgatgcggacctcaacaccggccacccgcaggtctacggcatgttccgcgatgaattcaccaacctgcgcagtcagtacggtgccggcggcttccgcttcgactttgttcggggctatgcgccggagcgggtcaacagctggatgaccgatagcgccgacaacagcttctgcgtcggcgaactgtggaaaggcccctctgagtacccgaactgggactggcgcaacaccgccagctggcagcagatcatcaaggactggtccgaccgggccaagtgcccggtgttcgacttcgccctcaaggaacgcatgcagaacgctcgatcgccgactggaagcacgcctgaacggcaatcccgacccgcgtggcgcgaggtggcggtgaccttcgtcgacaaccacgacaccggctactcgcccgggcagaacggtgggcagcaccactgggctctgcaggacgggctgatccgccaggcctacgcctacatcctcaccagccccggtacgccggtggtgtactggtcgcacatgtacgactggggttacggcgacttcatccgtcagctgatccaggtgcgtcgcgccgccggcgtgcgcgccgattcggcgatcagcttccacagcggctacagcggtctggtcgccaccgtcagcggcagccagcagaccctggtggtggcgctcaactccgacctgggcaatcccggccaggtggccagcggcagcttcagcgaggcggtcaacgccagcaacggccaggtgcgcgtgtggcgtagcggcacgggcagcggtggcggtgaacccggcgctctggtcagtgtgagtttccgctgcgacaacggcgcgacgcagatgggcgacagcgtctacgcggtcggcaacgtcagccagctcggtaactggagcccggccgcggcgttgcgcctgaccgacaccagcggctacccgacctggaagggcagcattgccttgcctgccggccagaacgaggaatggaaatgcctgatccgcaacgaggccaacgccacccaggtgcggcaatggcagggcggggcaaacaacagcctgacgccgagcgagggcgccaccaccgtcggccggctctag

(19) United States
(12) Patent Application Publication
(10) Pub. No.: US 2021/0120827 A1
(43) Pub. Date: Apr. 29, 2021

(54) BREAD QUALITY IMPROVING AGENT AND/OR QUALITY IMPROVING COMPOSITION

(71) Applicants: NAGASE CHEMTEX CORPORATION, Osaka-shi, Osaka (JP); HAYASHIBARA CO., LTD., Okayama-shi, Okayama (JP)

(72) Inventors: Naoki SHIRASAKA, Kyoto (JP); Koji UNO, Kyoto (JP); Yuri ARAKI, Kyoto (JP); Hisanori MIZOBUCHI, Kyoto (JP); Nariaki YOSHIDA, Okayama (JP)

(21) Appl. No.: 16/497,718

(22) PCT Filed: Mar. 26, 2018
(86) PCT No.: PCT/JP2018/012029

§ 371 Date: Sep. 25, 2019

(30) Foreign Application Priority Data
Mar. 27, 2017 (JP) 2017-061822

(51) Int. Cl. A21D 8/04; A21D 13/062; C12N 9/24

(52) U.S. Cl. CPC A21D 8/042; C12Y 302/0106; C12N 9/2402; A21D 13/062

(57) ABSTRACT

An object of the present invention is to improve the quality of bread by the action of an enzyme. The present invention provides a bread quality improver containing exomaltotetraohydrolase.

### TECHNICAL FIELD

The present invention relates to bread quality improvers and/or bread quality improving compositions.

### BACKGROUND ART

Staling of bread during storage is one of major problems in the bakery industry, and thus attempts have been made to prevent or delay such staling. In order to prevent staling of bread, it is conventional to add food additives such as enzymes, emulsifiers, oligosaccharides, or sugar alcohols during the preparation of dough. However, the bread produced with food additives other than enzymes is not consistent with the recent emphasis on using natural products as additives to food (Patent Literature 1).

Meanwhile, the sales of enzymes for industrial use in the Japanese domestic market are estimated to be about 26 billion yen, about 60% of which corresponds to enzymes for food. In the bread market, enzymes, which are natural products, have attracted increased attention as improvers, and many enzymes for breadmaking such as amylases and hemicellulases have been developed.

Exomaltotetraohydrolase (G4-producing enzyme) is an exoamylase that cleaves oligosaccharides as maltotetraose units from the nonreducing end of starch, and has been used as an enzyme for producing a maltooligosaccharide in the starch saccharification industry. Exomaltotetraohydrolase is known to act as a bread quality improver, e.g., to improve the elasticity and suppleness of baked bread and prevent hardening of baked bread. Exomaltotetraohydrolases derived from Bacillus circulans or Pseudomonas saccharophilia are well known (Patent Literatures 1 to 3).

### CITATION LIST

### Patent Literature

Patent Literature 1: JP H11-266773 A

Patent Literature 2: JP H11-178499 A

Patent Literature 3: JP 2007-526752 T

### SUMMARY OF INVENTION

### Technical Problem

Conventional enzyme-containing food improvers are insufficient to improve appearance, food texture, and flavor. An object of the present invention is to improve the quality of bread by the action of an enzyme.

### Solution to Problem

The present inventors made studies on the effects of enzymes on bread appearance, food texture, flavor, and saccharide composition, and found that exomaltotetraohydrolase may be used to improve the quality of bread. This finding has led to the completion of the present invention.

Specifically, the present invention relates to a bread quality improver, containing exomaltotetraohydrolase.

Preferably, the quality improver is for improving baked color.

Preferably, the quality improver is for improving food texture.

Preferably, the quality improver is for improving flavor.

Preferably, the quality improver is designed to cause production of saccharides mainly including maltose in bread dough.

Preferably, the saccharides mainly including maltose are produced by degradation by amylase of maltotetraose that is produced by the action of the exomaltotetraohydrolase.

Preferably, the exomaltotetraohydrolase is derived from Pseudomonas stutzeri.

The present invention also relates to a bread quality improving composition, containing the quality improver.

The present invention also relates to a method of producing bread, including adding the above composition to at least one bread dough ingredient to increase maltose content.

The present invention also relates to bread, produced by the method of producing bread.

The present invention also relates to bread, containing saccharides mainly including maltose.

### Advantageous Effects of Invention

The bread quality improver of the present invention which contains exomaltotetraohydrolase increases the maltose content in bread. Thus, the bread quality improver has advantageous effects on baked bread, including increasing volume, preventing staling (improving elasticity and suppleness of baked bread, and preventing hardening of baked bread or maintaining its softness), as well as providing a fresh baked color to bread, accelerating fermentation, improving food texture (reducing stickiness (kuchatsuki), improving springiness and melt-in-the-mouth texture), and improving moist texture and flavor (sweet taste, smell).

### BRIEF DESCRIPTION OF DRAWINGS

FIG. 1A shows the effect of the quality improving composition on the specific volume of bread.

FIG. 1B shows the effect of the quality improving composition on the specific volume of bread.

FIG. 2A shows the effect of the quality improving composition on the hardness of bread.

FIG. 2B shows the effect of the quality improving composition on the hardness of bread.

FIG. 3A shows the effect of the quality improving composition on the adhesiveness of bread.

FIG. 3B shows the effect of the quality improving composition on the adhesiveness of bread.

FIG. 4A shows the effect of the quality improving composition on the cohesiveness of bread.

FIG. 4B shows the effect of the quality improving composition on the cohesiveness of bread.

FIG. 5A shows the effect of the quality improving composition on the fragility of bread.

FIG. 5B shows the effect of the quality improving composition on the fragility of bread.

FIG. 6A shows the effect of the quality improving composition on the elasticity of bread.

FIG. 6B shows the effect of the quality improving composition on the elasticity of bread.

FIG. 7A shows the effect of the quality improving composition on the chewiness of bread.

FIG. 7B shows the effect of the quality improving composition on the chewiness of bread.

FIG. 8 shows the effect of the quality improving composition on the saccharide composition of bread.

FIG. 9 shows the effect of the quality improving composition on the total saccharide content of bread.

FIG. 10 shows the effects of the quality improving composition on the individual saccharide contents of bread.

FIG. 11A shows the effect of the quality improving composition on the taste and smell of bread.

FIG. 11B shows the effect of the quality improving composition on the taste and smell of bread.

FIG. 12A shows the appearance of a bread loaf produced with the quality improving composition.

FIG. 12B shows the appearance of a bread loaf produced with the quality improving composition.

FIG. 13A shows the appearance of a bread loaf produced with the quality improving composition.

FIG. 13B shows the appearance of a bread loaf produced with the quality improving composition.

FIG. 14A shows the effect of the quality improving composition on the specific volume of bread.

FIG. 14B shows the effect of the quality improving composition on the hardness of bread.

FIG. 14C shows the effect of the quality improving composition on the hardness of bread.

FIG. 14D shows the effect of the quality improving composition on the taste of bread.

FIG. 14E shows the effect of the quality improving composition on the saccharide composition of bread.

FIG. 14F shows the effect of the quality improving composition on the total saccharide content of bread.

FIG. 14G shows the effect of the quality improving composition on the baked color of bread.

FIG. 15A shows the effect of the quality improving composition on the specific volume of bread.

FIG. 15B shows the effect of the quality improving composition on the hardness of bread.

FIG. 15C shows the effects of the quality improving composition on the saccharide contents of bread.

FIG. 15D shows the effect of the quality improving composition on the taste of bread.

FIG. 16 shows the appearance of a croissant produced with the quality improving composition.

FIG. 17A shows the effect of the quality improving composition on the specific volume of bread.

FIG. 17B shows the effect of the quality improving composition on the hardness of bread.

FIG. 17C shows the effect of the quality improving composition on the taste of bread.

FIG. 18A shows the appearance of bread doughs during production by the sponge and dough method.

FIG. 18B shows the effect of the quality improving composition on the specific volume of bread.

FIG. 18C shows the effect of the quality improving composition on the hardness of bread.

FIG. 18D shows the effect of the quality improving composition on the hardness of bread.

FIG. 18E shows the effect of the quality improving composition on the saccharide composition of bread.

FIG. 18F shows the effect of the quality improving composition on the taste of bread.

### DESCRIPTION OF EMBODIMENTS

(1) Quality Improver

The bread quality improver of the present invention contains exomaltotetraohydrolase. Exomaltotetraohydrolase is known as an enzyme that causes exo-hydrolysis of starch and produces maltotetraose composed of four glucose molecules.

The exomaltotetraohydrolase may be derived from a microorganism, an animal, or a plant. Examples of the microorganism include those of the genus Pseudomonas or Bacillus. Examples of the microorganisms of the genus Pseudomonas include Pseudomonas stutzeri, Pseudomonas saccharophilia, and Pseudomonas sp. Examples of the microorganisms of the genus Bacillus include Bacillus circulans and Bacillus sp. Examples of the animal include mammals and reptiles. Examples of the mammals include pigs, rabbits, cattle, horses, wild boars, sheep, mice and rats, and hamsters. Examples of the reptiles include snakes. Examples of the plant include thale cress, peanuts, and cabbages. The exomaltotetraohydrolase is preferably derived from a microorganism, more preferably a microorganism of the genus Pseudomonas, still more preferably Pseudomonas stutzeri, among others. Also, the exomaltotetraohydrolase may be extracted from a microorganism, animal, or plant as an origin, or may be massively produced in microorganism cells. Genetically engineered exomaltotetraohydrolase may also be used, but non-genetically engineered (non-GMO) exomaltotetraohydrolase is preferred.

The exomaltotetraohydrolase is preferably any one of the following polypeptides (A), (B), and (C):

(A) a polypeptide containing the amino acid sequence of SEQ ID NO:1;

(B) a polypeptide having at least 85% sequence identity to the amino acid sequence of SEQ ID NO:1 and having the activity of causing exo-hydrolysis of starch to produce maltotetraose; and

(C) a polypeptide having an amino acid sequence obtained by deletion, insertion, substitution, and/or addition of one or more amino acids in the amino acid sequence of SEQ ID NO:1, and having the activity of causing exo-hydrolysis of starch to produce maltotetraose.

The sequence identity between the exomaltotetraohydrolase and the amino acid sequence of SEQ ID NO:1 is preferably at least 85%, more preferably at least 90%, still more preferably at least 95%, even more preferably at least 98%, particularly preferably at least 99%, most preferably 100%. Amino acid sequence identity refers to a value calculated by comparing the amino acid sequence to be evaluated with the amino acid sequence of SEQ ID NO:1 to determine the number of positions at which an identical amino acid occurs in both sequences, dividing the number of matched positions by the total number of amino acids, and multiplying the quotient by 100.

The number of amino acids deleted, inserted, substituted, and/or added is preferably 82 or smaller, more preferably 54 or smaller, still more preferably 27 or smaller, even more preferably 10 or smaller, particularly preferably 5 or smaller.

The exomaltotetraohydrolase is preferably a polypeptide encoded by any one of the following DNAs (a), (b), (c), and (d):

(a) a DNA that contains the base sequence of SEQ ID NO:2;

(b) a DNA that hybridizes under stringent conditions with a DNA containing a base sequence complementary to the base sequence of SEQ ID NO:2 and encodes a polypeptide having the activity of causing exo-hydrolysis of starch to produce maltotetraose;

(c) a DNA that has at least 85% sequence identity to the base sequence of SEQ ID NO:2 and encodes a polypeptide having the activity of causing exo-hydrolysis of starch to produce maltotetraose; and

(d) a DNA that has a base sequence obtained by deletion, insertion, substitution, and/or addition of one or more bases in the base sequence of SEQ ID NO:2 and encodes a polypeptide having the activity of causing exo-hydrolysis of starch to produce maltotetraose.

The DNA that hybridizes under stringent conditions with a DNA containing a base sequence complementary to the base sequence of SEQ ID NO:2 and encodes a polypeptide having the activity of causing exo-hydrolysis of starch to produce maltotetraose refers to a DNA which may be obtained by a technique such as colony hybridization, plaque hybridization, or southern hybridization under stringent conditions using a DNA having a base sequence complementary to the base sequence of SEQ ID NO:2 as a probe, and which encodes a polypeptide having the activity of causing exo-hydrolysis of starch to produce maltotetraose.

The hybridization can be accomplished by known methods. The DNA that hybridizes under stringent conditions may refer to a DNA obtained, for example, by performing hybridization using a filter with a colony- or plaque-derived DNA immobilized thereon in the presence of 0.7 to 1.0 M NaCl at 65° C., and then washing the filter at 65° C. with a 2×SSC solution (the composition of a 1×SSC solution is as follows: 150 mM sodium chloride and 15 mM sodium citrate). It is preferably a DNA obtained by washing at 65° C. with a 0.5×SSC solution, more preferably a DNA obtained by washing at 65° C. with a 0.2×SSC solution, still more preferably a DNA obtained by washing at 65° C. with a 0.1×SSC solution.

The sequence identity between the DNA encoding the exomaltotetraohydrolase and the base sequence of SEQ ID NO:2 is preferably at least 85%, more preferably at least 90%, still more preferably at least 95%, even more preferably at least 98%, particularly preferably at least 99%, most preferably 100%.

Base sequence identity refers to a value calculated by comparing the base sequence to be evaluated with the base sequence of SEQ ID NO:2 to determine the number of positions at which an identical base occurs in both sequences, dividing the number of matched positions by the total number of bases, and multiplying the quotient by 100.

The DNA that has a base sequence obtained by deletion, insertion, substitution, and/or addition of one or more bases in the base sequence of SEQ ID NO:2 and encodes a polypeptide having the activity of causing exo-hydrolysis of starch to produce maltotetraose can be prepared according to known gene modification methods.

The number of bases deleted, inserted, substituted, and/or added is preferably 246 or smaller, more preferably 164 or smaller, still more preferably 82 or smaller, even more preferably 32 or smaller, particularly preferably 16 or smaller.

The amino acid sequence of SEQ ID NO:1 and the base sequence of SEQ ID NO:2 are the amino acid sequence of exomaltotetraohydrolase of Pseudomonas stutzeri MO-19 and the base sequence of the gene thereof, respectively.

When the enzyme protein exomaltotetraohydrolase is added to bread dough, the exomaltotetraohydrolase in the dough will undergo thermal denaturation and lose its function as the dough temperature increases during heating. The exomaltotetraohydrolase can be digested and absorbed in the body similarly to the proteins contained in eggs or other ingredients.

The exomaltotetraohydrolase can be prepared from naturally occurring organisms. When the exomaltotetraohydrolase is prepared from a naturally occurring microorganism, the preparation may be carried out by a method including culturing a microorganism capable of producing exomaltotetraohydrolase, separating the microorganism cells from the culture liquid, and purifying the exomaltotetraohydrolase.

In the step of culturing a microorganism capable of producing exomaltotetraohydrolase, the microorganism is cultured in a culture medium containing nutrient sources that can be utilized by the microorganism. The medium may be in liquid or solid form as long as it accelerates the production of exomaltotetraohydrolase. For mass culture of the microorganism, it is preferred to use a liquid medium because such a medium is easy to prepare, and it is stirrable and allows for culturing at a high microbial concentration.

Examples of the nutrient sources include carbon sources, nitrogen sources, and inorganic salts. Examples of the carbon sources include glucose, glycerol, dextrin, starches, molasses, and animal and vegetable oils. Examples of the nitrogen sources include soy flour, corn steep liquor, cottonseed meal, meat extract, peptone, yeast extract, ammonium sulfate, sodium nitrate, and urea. Examples of the inorganic salts include sodium, potassium, calcium, magnesium, manganese, iron, cobalt, zinc, and phosphates. The culturing may be carried out under static, shaking, or aerated conditions. For mass culture of the microorganism, aerated culture conditions are preferred because air and nutrient sources can be efficiently supplied to the cells.

The culture temperature is preferably 10° C. to 60° C., more preferably 20° C. to 40° C. The pH of the medium is preferably 5 to 9. The culture duration is, for example, one to seven days; the culture liquid may be monitored by a method commonly used by a person skilled in the art, and the culturing may be terminated when the exomaltotetraohydrolase content in the culture liquid reaches the maximum.

In the step of separating the microorganism cells from the culture liquid, the microorganism cells can be separated from the culture liquid by, for example, centrifugation, filtration, or reduced pressure distillation.

In the step of isolating and purifying the exomaltotetraohydrolase from the liquid containing exomaltotetraohydrolase, known techniques including ultrafiltration or microfiltration using a filter membrane having a molecular weight cut-off of 60000 or less, fractionation using ammonium sulfate or ethanol, and purification by chromatography can appropriately be used in combination according to the desired degree of purification of the exomaltotetraohydrolase.

The exomaltotetraohydrolase content in the quality improver is not limited, but is 0.5 U to 750000 U, preferably 1 U to 720000 U, more preferably 5 U to 700000 U, still more preferably 10 U to 100000 U, even more preferably 100 U to 10000 U, most preferably 6000 U to 8000 U, per gram of the total weight of the quality improver. Here, the activity of the exomaltotetraohydrolase may be measured by allowing the enzyme to act on the substrate starch and quantitating the reducing power of the produced reducing sugar by the Somogyi-Nelson method. The enzyme activity is expressed in units, where 1 U is defined as the amount of the enzyme required to produce a reducing power corresponding to 1 μmol of glucose per minute.

The bread quality improver of the present invention may consist only of exomaltotetraohydrolase or may contain exomaltotetraohydrolase and additional components that can be generally used in enzyme preparations as long as the components do not inhibit the effects of the present invention. Examples of such components include excipients, pH adjusters, and preservatives.

One skilled in the art can select an appropriate excipient or optionally a combination of such excipients. Examples of the excipients include, but are not limited to, dextrin and trehalose.

Examples of the pH adjusters include ascorbic acid (vitamin C), acetic acid, dehydroacetic acid, lactic acid, citric acid, gluconic acid, succinic acid, tartaric acid, fumaric acid, malic acid, and adipic acid, and sodium (Na), calcium (Ca), or potassium (K) salts of these organic acids; and carbonic acid, phosphoric acid, and pyrophosphoric acid, and Na or K salts of these inorganic acids.

In addition to adjusting the pH, ascorbic acid (vitamin C) also contributes to an increase in the volume of bread. In other words, ascorbic acid may come into contact with oxygen so that it can be oxidized in bread dough. The oxidized ascorbic acid acts on the gluten in the wheat flour to tighten the bread dough, thereby preventing stickiness of the bread dough. Moreover, tightening the bread dough permits the dough to keep CO₂, thereby accelerating an increase in bread volume.

The content of additional components other than the exomaltotetraohydrolase in the bread quality improver is not particularly limited. For example, when a pH adjuster such as sodium ascorbate is added, its content is preferably 0.1 to 100 ppm, more preferably 5 to 60 ppm, further preferably 10 to 50 ppm, most preferably 20 to 40 ppm, of the content of wheat flour which is an ingredient of bread.

Examples of the preservatives include propionic acid, propionate salts, sulfite salts, benzoate salts, sorbic acid, and sorbate salts. Examples of the salts include sodium (Na), calcium (Ca), and potassium (K) salts.

When a bread quality improver is prepared by mixing exomaltotetraohydrolase with additional components, the exomaltotetraohydrolase may be mixed with an excipient in a mixer such that the above activity value is obtained. Examples of the mixer include rotary vessel mixers, stationary vessel mixers, and complex type mixers, and an appropriate mixer can be selected according to the target activity value or amount, or the type of excipient.

As described later, the exomaltotetraohydrolase in the present invention produces maltotetraose, which is then degraded by the amylase present in the ingredient wheat flour to produce maltose. Thus, the quality improver is preferably designed to cause production of saccharides mainly including maltose in bread dough. Moreover, the saccharides mainly including maltose are preferably produced by degradation by amylase of maltotetraose that is produced by the action of the exomaltotetraohydrolase.

(2) Quality Improving Composition

The bread quality improving composition of the present invention is characterized by containing the quality improver. The quality improving composition may contain the quality improver and additional components acceptable in food.

Examples of additional components other than the exomaltotetraohydrolase which can be used in the bread quality improving composition of the present invention include enzymes, thickening polysaccharides, emulsifiers, mixtures of emulsifiers and polyphosphates, dairy products, extracts, saccharides, sweeteners, fermented seasonings, eggs, and inorganic salts. The content of additional components in the bread quality improving composition of the present invention is not particularly limited, and one skilled in the art can select any appropriate content.

Examples of the enzymes include α-amylase, β-amylase, maltogenic amylase, glucan 1,4-α-maltotriohydrolase, glucan 1,4-α-maltohexaohydrolase, hemicellulase, phospholipase, galactolipase, glucose oxidase, ascorbic acid oxidase, peroxidase, catalase, glutathione dehydrogenase, protease, peptidase, transglutaminase, cyclodextrin glucanotransferase, β-glucanase, triacylglycerol lipase, and chitinase.

Examples of the thickening polysaccharides include modified starches, gums, alginic acid, alginic acid derivatives, pectin, carrageenan, curdlan, pullulan, gelatin, cellulose derivatives, agar, tamarind, psyllium, and glucomannan.

Examples of the emulsifiers include glycerol fatty acid esters, polyglycerol fatty acid esters, sucrose fatty acid esters, propylene glycol fatty acid esters, sorbitan fatty acid esters, lecithin, enzymatically decomposed lecithin, and saponin.

Examples of the dairy products include milk, skim milk powder, whole milk powder, whey powder, casein, cheese, yogurt, condensed milk, fermented milk, and cream.

Examples of the extracts include yeast extract and malt extract.

Examples of the saccharides include monosaccharides such as glucose and fructose; disaccharides such as sucrose, maltose, isomaltose, trehalose, lactose, lactulose, and cellobiose; linear or branched oligosaccharides such as maltotriose and higher maltooligosaccharides, raffinose, panose, stachyose, glucooligosaccharides, maltooligosaccharides, isomaltooligosaccharides, fructooligosaccharides, xylooligosaccharides, soybean oligosaccharides, gentioligosaccharides, nigerooligosaccharides, galactooligosaccharides, mannanoligosaccharides, and lactosucrose; sugar mixtures such as isomerized sugar, starch syrup, powdered starch syrup, and honey; polysaccharides such as starches, modified starches, dextrin, and hydroxylated hemicellulose; and sugar alcohols such as reduced starch syrup, maltitol, lactitol, sorbitol, mannitol, xylitol, palatinit, erythritol, and reduced oligosaccharides. Disaccharides, oligosaccharides, starches, modified starches, and dextrin can also be used as excipients.

Examples of the sweeteners include stevia, aspartame, glycyrrhizin, acesulfame potassium, sucralose, and neotame.

Examples of the inorganic salts include sodium chloride, ammonium sulfate, sodium sulfate, calcium chloride, and polymerized phosphates.

The bread quality improving composition of the present invention may be in any form, such as, for example, powder, granules, liquid, paste, or solid. In the case of a powdery bread quality improving composition, the powder form may be obtained by dissolving exomaltotetraohydrolase in a solvent such as water or a sugar solution, adding an optional excipient such as dextrin, and drying the mixture.

The amount of the quality improver in the bread quality improving composition is preferably 0.1 to 10%, more preferably 1 to 5%, still more preferably 2 to 3%. The symbol “%” means weight/weight percent, unless otherwise specified.

(3) Details of Quality Improvement

Adding the bread quality improving composition of the present invention to bread ingredients enables improvement in the quality of baked bread. The quality improvement points include improvements in baked color, food texture, and flavor.

A specific example of improvement in the baked color of bread is providing a fresh baked color. The baked color can be evaluated by measuring the color difference of bread.

Specific examples of improvements in food texture include reduction in stickiness (kuchatsuki), improvement in springiness, and improvement in melt-in-the-mouth texture. The stickiness (kuchatsuki) can be evaluated by measuring the adhesiveness of bread and sensory testing. The springiness can be evaluated by measuring the elasticity of bread and sensory testing. The melt-in-the-mouth texture can be evaluated by measuring the cohesiveness, fragility, and chewiness of bread and sensory testing.

Specific examples of improvements in flavor include improvements in sweetness and smell. The flavor can be evaluated by sensory testing.

The other quality improvement points include an increase in the volume of bread, prevention of staling, and acceleration of fermentation. The increase in volume can be evaluated by measuring the specific volume of bread. The prevention of staling can be achieved by improvements in the elasticity and suppleness of bread and prevention of hardening. The prevention of staling can be evaluated by measuring the specific volume, hardness, cohesiveness, fragility, elasticity, and chewiness of bread and sensory testing. The acceleration of fermentation can be evaluated by measuring the specific volume. The following describes the evaluation items.

Specific volume: The bread quality improving composition of the present invention acts on the starch in wheat flour to produce oligosaccharides such as maltose. This accelerates fermentation of bakery yeast, thereby increasing the specific volume of baked bread. The increase in specific volume leads to a volume-increasing effect and an anti-staling effect.

The specific volume may be determined by measuring the volume (cm³) and weight (g) of bread to calculate the specific volume (cm³/g). The volume and weight may be measured with a laser volume meter. The specific volume of bread produced with the bread quality improving composition of the present invention is preferably at least 1.04 times, more preferably at least 1.08 times, still more preferably at least 1.1 times, even more preferably at least 1.15 times, the specific volume of bread produced under the same conditions except that no bread quality improving composition of the present invention is added.

Color difference: The bread produced with the bread quality improving composition of the present invention has a darker baked color due to the increase in maltose content. The baked color of bread is produced as the saccharides in the bread develop a color through a Maillard reaction and a caramelization reaction. The saccharide most likely to undergo a Maillard reaction is fructose, followed by glucose, maltose, lactose, and sucrose, in decreasing order of likelihood. The exomaltotetraohydrolase in the bread quality improving composition of the present invention produces maltotetraose, which is then degraded by the amylase present in the ingredient wheat flour to produce maltose. The increase in maltose content results in a fresh baked color. Baked color is a key factor for bread.

The term “color difference” means a difference in color between two samples (color stimuli) as defined using ΔL*, Δa*, and Δb*, which are the differences in the coordinates L*, a*, and b*, respectively, in the L*a*b* color system. The bread produced with the bread quality improving composition of the present invention preferably has a color difference at the same portion of at least 3.5, more preferably at least 4.0, still more preferably at least 5.0, even more preferably at least 7.0, compared to the bread produced under the same conditions except that no bread quality improving composition of the present invention is added.

Hardness: The bread quality improving composition of the present invention moderately degrades starch to reduce recrystallization of the starch. Also, the increase in maltose content increases moisture retaining properties. The resulting bread keeps its softness and is prevented from staling. The hardness may be measured as the maximum test force (N) when stress is applied to bread using a plunger of a rheometer. The bread produced with the bread quality improving composition of the present invention preferably has a hardness at the same portion of 0.93 times or less, more preferably 0.85 times or less, still more preferably 0.8 times or less, even more preferably 0.73 times or less, the hardness of the bread produced under the same conditions except that no bread quality improving composition of the present invention is added.

Adhesiveness: The bread quality improving composition of the present invention moderately degrades starch to reduce excessive gelatinization. Also, the increase in maltose content increases moisture retaining properties. The resulting bread has reduced adhesiveness and stickiness (kuchatsuki).

Cohesiveness: It is generally thought that the use of bread quality improvers may increase cohesiveness. However, the bread quality improving composition of the present invention reduces the increase in cohesiveness. Thus, the bread quality improving composition of the present invention prevents staling of bread and improves the melt-in-the-mouth texture of bread.

Fragility: The bread quality improving composition of the present invention moderately degrades starch to reduce recrystallization of the starch. Also, the increase in maltose content increases moisture retaining properties. This therefore contributes to an increase in fragility of bread to prevent staling of the bread and improve the melt-in-the-mouth texture. The fragility may be defined as the value (N) determined when stress is applied to the crumb of Pullman bread using a plunger of a rheometer. The bread produced with the bread quality improving composition of the present invention preferably has a fragility at the same portion of 0.86 times or less, more preferably 0.8 times or less, still more preferably 0.75 times or less, the fragility of the bread produced under the same conditions except that no bread quality improving composition of the present invention is added.

Elasticity: The bread quality improving composition of the present invention moderately degrades starch to reduce excessive gelatinization, thereby maintaining the elasticity of bread. The resulting bread is prevented from staling and has improved springiness.

Chewiness: The bread quality improving composition of the present invention moderately degrades starch to reduce recrystallization of the starch, thereby improving chewiness. Also, the increase in maltose content increases moisture retaining properties. The resulting bread is prevented from staling and has an improved melt-in-the-mouth texture. The chewiness is given by the relationship hardness (N)×elasticity×cohesiveness, and these values may be measured using a rheometer. The bread produced with the bread quality improving composition of the present invention preferably has a chewiness of 0.8 times or less, more preferably 0.75 times or less, the chewiness of the bread produced under the same conditions except that no bread quality improving composition of the present invention is added.

Saccharide composition: The bread produced by conventional methods contains fructose as a main saccharide component. In contrast, in the present invention, the exomaltotetraohydrolase produces maltotetraose, which is then degraded by the amylase present in the ingredient wheat flour to produce maltose. The term “main saccharide component” refers to the component that constitutes the highest proportion in the saccharide composition of bread. The increase in maltose content improves specific volume, color difference, food texture, and flavor.

The proportion of maltose in the saccharide composition of bread is not particularly limited as long as maltose is the main component, and the proportion may vary depending on the ingredients used. The proportion is preferably 15 to 80%, more preferably 20 to 60%, still more preferably 25 to 40%, even more preferably 30 to 40%.

The saccharide composition of baked bread and of bread dough can be measured as follows by ordinary methods. For example, in order to measure the saccharide composition of bread dough, the components extracted from the bread dough with water may be analyzed by HPLC. The method of extraction from bread dough with water and the HPLC analysis conditions are as follows.

(Method of Extraction from Bread Dough with Water)
(1) Frozen dough is partially thawed.
(2) In a 50-mL beaker, 10 g of frozen dough or baked dough and 30 g (in the case of a French bread, 40 g) of 30 mM HCl are mixed.
(3) The mixture from the step (2) is mixed using a stirrer for 90 minutes to 2 hours to cause extraction.
(4) The whole amount is transferred to a 50-mL centrifuge tube, followed by centrifugation at 8000 rpm for 30 minutes.
(5) The supernatant is weighed out into an Eppendorf tube, followed by centrifugation at 14000 rpm for 30 to 60 minutes.
(6) The supernatant is filtrated through a filter and subjected to analysis by HPLC.

(HPLC Analysis Conditions)
Column: Xbridge Amide 4.6×150 mm
Mobile phase: 77% aqueous acetone solution+0.05% triethylamine (v/v), pH 10.3
Column temperature: 85° C.
Flow rate: 0.5 mL/min
Detector: RI
Maximum pressure: 40 MPa
Acceptable pH: pH 2 to 11

Saccharide content: The bread produced with the bread quality improving composition of the present invention has a higher maltose content and a higher total saccharide content, which improves baked color, food texture, and flavor.

The maltose content in the whole bread is preferably 0.6 to 20%, more preferably 1 to 10%, still more preferably 1.2 to 5%, although it depends on the ingredients used. The total saccharide content in the whole bread is preferably 4 to 25%, more preferably 4 to 16%, still more preferably 4 to 7%, although it depends on the ingredients used.

The bread produced with the bread quality improving composition of the present invention has a maltose content that is preferably at least 1.5 times, more preferably at least 2 times, still more preferably at least 2.5 times, even more preferably at least 2.8 times, the maltose content of the bread produced under the same conditions except that no bread quality improving composition of the present invention is added.

The saccharide content of bread can be measured as follows by an ordinary method such as the anthrone-sulfuric acid method. In order to measure the saccharide content by the anthrone-sulfuric acid method, the measurement method is as follows.
(1) 150 mL of sulfuric acid is mixed, while cooling, into 50 mL of distilled water, and 0.4 g of anthrone is dissolved in the mixture to prepare a 0.2% anthrone solution.
(2) A sugar solution sample is diluted with distilled water.
(3) 0.4 mL of the sugar solution is mixed with 2 mL of the cooled anthrone solution.
(4) The mixture is heated in a boiling water bath for 10 minutes, followed by cooling.
(5) The absorbance at 620 nm is measured. The saccharide concentration of the diluted solution is determined based on a calibration curve prepared using standards. Based on the dilution ratio in the step (2), the saccharide content is determined.

Food texture: The bread produced with the bread quality improving composition of the present invention has a high maltose content. Since maltose increases moisture retaining properties, the food texture can be improved. The improvements in food texture include reduction in stickiness (kuchatsuki), improvement in springiness, and improvement in melt-in-the-mouth texture. Moreover, since maltose is different in flavor from glucose, the flavor can be improved. The flavor may be improved in terms of moist texture, sweetness, and smell.

Smell: The bread quality improving composition of the present invention is excellent in accelerating fermentation, so that the bread dough containing the composition gives off a strong alcohol smell while being baked. Meanwhile, the bread produced with the composition has a strong sweet aroma due to the high content of saccharides mainly including maltose, which masks the alcohol smell. Thus, the baked bread has a reduced alcohol smell and produces a sweet aroma.

(4) Method of Producing Bread

The method of producing bread according to the present invention includes adding the composition to at least one bread dough ingredient to increase the maltose content in baked bread. When the composition is added to bread dough ingredients and the dough is fermented, the starch in the dough ingredients is degraded by the activity of exomaltotetraohydrolase to produce maltotetraose, which is then degraded by the amylase present in the ingredient wheat flour, so that the maltose content is increased. The increase in maltose content provides an improved quality to baked bread. The increase in maltose content may occur either in the bread dough ingredients or in the baked bread.

Examples of the ingredients of the bread dough include wheat flour (e.g., soft flour, medium flour, strong flour, whole wheat flour, graham flour), yeast (e.g., fresh yeast, dry yeast, instant dry yeast), sugars (e.g., table sugars such as caster sugar, granulated sugar, soft brown sugar, and brown sugar, isomerized sugar, powdered starch syrup, starch syrup, sugar alcohol, oligosaccharide, trehalose), table salt, dairy ingredients (e.g., milk, cream, whole milk powder, skim milk powder, milk protein, concentrated milk), oils and fats (e.g., shortening, margarine, butter, liquid oil, emulsified oils and fats), water, eggs (e.g., whole egg, egg yolk, egg white, dried egg, frozen egg), and baking powder.

In order to give variety to the flavor, taste, and food texture, other ingredients may be added, such as grain flour other than wheat flour (e.g., rice flour, rye flour, corn starch, soy flour); dairy products such as milk, dairy cream, yogurt, cream cheese, and sour cream; chocolates; powder ingredients such as cocoa powder, coffee powder, matcha green tea powder, and black tea powder; spices and herbs such as cinnamon and vanilla beans; fruit juice, fruits, nuts, alcohol, and flavorings.

The bread quality improving composition of the present invention can be added to the bread dough ingredients by any method. The quality improving composition of the present invention may be added or incorporated before, during, or after the mixing process in bread production. It is preferred to mix the bread dough ingredients with the quality improving composition. Here, the quality improving composition may be added either before or during the mixing process. The term “mixing” means mixing and kneading the bread dough ingredients with the quality improving composition of the present invention. The mixing can be performed under ordinary conditions employed in bread production.

The quality improving composition may be directly added to any of the bread dough ingredients or may be previously dissolved in liquid such as water, followed by adding it to the bread dough ingredients. Moreover, the quality improving composition may be mixed with the whole bread dough ingredients, or may be mixed with some of the bread dough ingredients, e.g., wheat flour, and then with the other bread dough ingredients. For example, in the case where the quality improving composition of the present invention is in the form of powder, the composition may be powder-mixed (preferably mixed and sieved) with powdery ingredients. The quality improving composition of the present invention may optionally be dissolved (in the case of powder form) or diluted (in the case of liquid form) in water together with table salt or sugar. The quality improving composition of the present invention may optionally be previously incorporated or dispersed and dissolved into an oil or fat such as margarine before use.

The bread dough can be produced and baked by ordinary methods. Examples of such bread dough production methods include the sponge and dough method (sponge method), the straight dough method, the refrigerated dough method, the frozen dough method, the Poolish dough method, the sourdough method, the sakadane (sake yeast) method, the hop yeast method, the soaker dough method, the Chorleywood bread process, and the continuous bread-making method.

In the sponge and dough method, part or whole of wheat flour is fermented first to prepare a sponge, and then the rest of the wheat flour and ingredients are added to the sponge, followed by mixing to make a final dough. In the sponge and dough method, the bread quality improving composition can produce its effect when incorporated into either the sponge ingredients or the final dough ingredients, but the composition is preferably incorporated into the sponge ingredients. In the present invention, since the maltotetraohydrolase functions during fermentation, a preferred method of producing bread dough is the sponge and dough method which allows for a long enzyme reaction time.

For example, bread may be produced as follows by the sponge and dough method. The sponge ingredients are mixed and fermented, e.g., at 25° C. to 35° C. for 2 to 5 hours (sponge fermentation). The sponge is mixed with the final dough ingredients. The resulting bread dough is typically allowed to rest at 15° C. to 35° C. for 10 to 40 minutes (floor time). Then, the dough is appropriately divided into pieces suited for a desired bread shape and allowed to rest, e.g., at 15° C. to 35° C. for 10 to 30 minutes (bench rest time). The pieces are shaped and subjected to final fermentation, e.g., at 25° C. to 45° C., until the dough pieces expand to an appropriate size, followed by baking at 160° C. to 250° C. for 10 to 60 minutes, whereby bread loaves are produced.

In the straight dough method, dough is prepared by mixing all the ingredients from the beginning and fermenting the mixture. In the straight dough method, the bread quality improving composition is preferably incorporated together with the other ingredients from the beginning.

For example, bread can be produced as follows by the straight dough method. The quality improving composition of the present invention is mixed with the bread dough ingredients to obtain bread dough. The dough is fermented, e.g., at 25° C. to 40° C. for 30 to 120 minutes (primary fermentation). Then, if necessary, the bread dough is appropriately divided into pieces suited for a desired bread shape and the pieces are shaped and further fermented, e.g., at 25° C. to 45° C. (for example, for 30 to 150 minutes), until the dough pieces expand to an appropriate size. After the fermentation, the pieces are heated (e.g., baked) at 160° C. to 250° C. for 10 to 60 minutes, whereby bread loaves are produced.

In the refrigerated dough method, dough is produced by the same procedure as in the sponge and dough method or the straight dough method. This method is characterized in that the dough is refrigerated and stored once in any subsequent step. In the case where a sponge is produced, the sponge may be refrigerated. In the refrigerated dough method, when dough is produced by the same procedure as in the sponge and dough method, the bread quality improving composition can produce its effect when incorporated into either the sponge ingredients or the final dough ingredients, but the composition is preferably incorporated into the sponge ingredients. In the case where dough is produced by the same procedure as in the straight dough method, the bread quality improving composition is preferably incorporated together with the other ingredients from the beginning.

In the frozen dough method, dough is produced by the same procedure as in the sponge and dough method or the straight dough method. This method is characterized in that the dough is frozen and stored once in any subsequent step. In the frozen dough method, when dough is produced by the same procedure as in the sponge and dough method, the bread quality improving composition can produce its effect when incorporated into either the sponge ingredients or the final dough ingredients, but the composition is preferably incorporated into the sponge ingredients. In the case where dough is produced by the same procedure as in the straight dough method, the bread quality improving composition is preferably incorporated together with the other ingredients from the beginning.

The freezing process may be carried out by holding the bread dough at a temperature of −80° C. to −10° C. The temperature conditions may be constant or may appropriately vary. In the case of varying temperature conditions, for example, the dough may be held at −40° C. to −30° C. for about 1 to 3 hours and then at −20° C. to −10° C. for a few days to several months, but the conditions are not limited thereto. The freezing time may be adjusted as appropriate depending on the type or size of bread or the desired storage period.

When the bread dough is frozen, it is preferably then thawed before use in production. The thawing process may be carried out by holding the bread dough at, for example, 15° C. to 30° C. until it is completely thawed.

In the Poolish dough method which is characterized in that a fermentation product of bakery yeast is previously produced in liquid, dough is produced by the same procedure as in the sponge and dough method. In the Poolish dough method, the bread quality improving composition can produce its effect when incorporated into either the sponge ingredients or the final dough ingredients, but the composition is preferably incorporated into the sponge ingredients.

In the other methods, some of the ingredients and steps may be different from those described above. However, in all these methods, the effects of the present invention can be achieved by incorporating the quality improving composition during the production of fermented starter dough.

The term “fermentation” means that the yeast present in the bread dough ingredients produces carbon dioxide gas and metabolites, so that the bread dough expands and its flavor is improved. In bread production, the bread dough obtained in the mixing process is preferably subjected to a fermentation process. Herein, the term “fermentation process” refers to being actively subjected to an environment where fermentation proceeds.

The temperature during the bread dough fermentation process may be any condition employed in an ordinary bread making method. The temperature may be selected appropriately depending on the type of bread, but is preferably 0° C. to 45° C., more preferably 25° C. to 45° C., still more preferably 35° C. to 38° C.

The humidity during the bread dough fermentation process may be any condition employed in an ordinary bread making method. The humidity may be selected appropriately depending on the type of bread, but is preferably 50 to 95%, more preferably 70 to 95%, still more preferably 80 to 90%.

The duration of the bread dough fermentation process may be any condition employed in an ordinary bread making method. The duration may be selected appropriately depending on the type of bread, but is preferably 0 to 20 hours, more preferably 0 to 4 hours, still more preferably 50 to 100 hours. The fermentation duration means the duration of final fermentation after shaping.

The quality improving composition content in the bread dough can be selected as appropriate according to the conditions employed in the particular bread making method used. For example, the content is preferably 50 to 400 ppm (222 to 1776 U/kg of strong flour), more preferably 100 to 300 ppm (444 to 1332 U/kg of strong flour), still more preferably 150 to 250 ppm (666 to 1110 U/kg of strong flour). Here, 1 U is a unit representing the activity of exomaltotetraohydrolase described above.

The bread dough may be heated by baking or steaming. The heating temperature for the bread dough may be any condition employed in an ordinary bread making method. The heating temperature may be selected appropriately depending on the type of bread, but is preferably 170° C. to 250° C., more preferably 190° C. to 220° C., in the case of heating by baking, and is preferably 100° C. to 140° C., more preferably 115° C. to 125° C., in the case of heating by steaming.

The heating duration for the bread dough may be any condition employed in a common bread making method. The heating duration may be selected appropriately depending on the type of bread, but is preferably 10 to 70 minutes, more preferably 15 to 60 minutes, still more preferably 20 to 50 minutes, even more preferably 20 to 40 minutes.

Further, the bread may be stuffed with a filling, or the surface thereof may be covered with a spread. Examples of such fillings and spreads include custard cream, chocolate cream, jams, paste, and prepared foods (e.g., curry, stir-fried noodles, tuna, egg, potato).

Examples of breads to which the quality improver of the present invention is applicable include white breads, healthy breads, sweet breads (e.g., sweet bean buns, jam buns, cream buns), bread rolls, French breads, steamed breads, savory breads, hot dog buns, fruit breads, cornbreads, butter rolls, buns, sandwiches, croissants, danish pastries, hard biscuits, bagels, and pretzels. Among these, sweet breads such as sweet bean buns, jam buns, and cream buns, and butter rolls are collectively called variety breads.

The present invention also relates to a method of improving the quality of bread, including adding the quality improving composition to at least one bread dough ingredient. The bread quality improving composition can be added to the bread dough ingredient by any method. The quality improving composition may be added or incorporated into the bread dough ingredients before, during, or after the mixing process in bread production. It is preferred to mix the bread dough ingredients with the quality improving composition. In this case, the quality improving composition may be added either before or during the mixing process.

The present invention also relates to a method of adjusting the maltose content in bread, including adjusting the maltose content in baked bread to 0.6 to 20% without adding maltose to bread dough ingredients. The maltose content is preferably 0.6 to 20%, more preferably 1 to 10%, still more preferably 1.2 to 5%, although it depends on the ingredients used. This method can adjust the maltose content without adding maltose to bread dough ingredients, thereby improving the quality of bread. The details of bread quality improvement are as described above for the bread quality improver.

The present invention also relates to a maltose content adjusting agent for use in the above adjustment method and a maltose content adjusting composition containing the adjusting agent.

The maltose content adjusting agent may contain exomaltotetraohydrolase and additional components that can be generally used in enzyme preparations. The additional components may be the components described above for the bread quality improver.

The maltose content adjusting composition may contain the maltose content adjusting agent and additional components acceptable in food. The additional components may be the components described above for the bread quality improving composition.

The present invention also relates to bread produced through the method of improving the quality of bread or the method of adjusting the maltose content in bread dough ingredients. The present invention also relates to bread containing saccharides mainly including maltose. The method of producing the bread and the type of bread are as described above.

### EXAMPLES

### Example 1

Exomaltotetraohydrolase (enzyme powder) and a food ingredient (dextrin) were mixed such that the exomaltotetraohydrolase content was about 3% and the food ingredient content was about 97%. The mixture was pulverized to prepare a powdery bread quality improving composition.

### Example 2 and Comparative Examples 1 to 3

White bread loaves were produced with the bread quality composition of Example 1 using a sponge and dough recipe (Example 2). Also, bread loaves were produced with no enzyme (Comparative Example 1), or using maltogenic amylase (Bakezyme MA 10000, DSM) (Comparative Example 2) or α-amylase (Bakezyme P500, DSM) (Comparative Example 3) in place of the bread quality improving composition of Example 1.

Table 1 shows the ingredient contents.

TABLE 1

| | Sponge | Final dough |
|---|---|---|
| Wheat flour (strong flour) | 70% | 30% |
| Bakery yeast | 2.5% | — |
| Quality improving composition | Given amount | — |
| Table salt | — | 2% |
| Sugar | — | 6% |
| Skim milk powder | — | 3% |
| Shortening | — | 5% |
| Water | 40% | 28% |

Each ingredient content in Table 1 is expressed as parts by weight based on 100 parts by weight of wheat flour in the final bread dough after mixing of the final dough ingredients. The quality improving composition (mixture of exomaltotetraohydrolase and dextrin) content was 200 ppm in Example 2, 50 ppm for maltogenic amylase (Comparative Example 2), and 5 ppm for α-amylase (Comparative Example 3).

The sponge ingredients indicated in Table 1 were mixed, and a sponge was produced in the step shown in Table 2.

TABLE 2

| Sponge step | Conditions |
|---|---|
| Mixing duration | Low speed, 2 min - |
| | low-mid speed, 2 min |
| Final dough temperature | 24° C. |
| (° C.) | |
| Fermentation duration | 4 h |
| Fermentation | 28° C., 80% |
| temperature, humidity | |

The produced sponge was mixed with the final dough ingredients indicated in Table 1, and the final dough was baked in the step shown in Table 3 to produce bread loaves.

TABLE 3

| Final dough step | Conditions |
|---|---|
| Mixing duration | Low speed, 1 min - low-mid speed, 3 min - |
| | mid-high speed, 1 min |
| | ↓ |
| | Low-mid speed, 3 min - mid-high speed, 1 |
| | min |
| Final dough temperature | 27° C. |
| (° C.) | |
| Fermentation duration | 15 min |
| Fermentation temperature, | 28° C., 80% |
| humidity | |
| Divided weight | 1. Round-top: 300 g |
| | 2. Pullman: 210 g × 3 |
| Bench rest time (min) | 15 min |
| Shaping conditions | Moulder gap |
| Proofing temperature, | 35° C., 85% |
| humidity | |
| Proofing time | 1. Round-top: 1.5 cm above pan |
| | 2. Pullman: 80% of pan |
| Baking temperature, time | 1. Round-top: upper heat 195° C./lower |
| | heat 210° C.: 25 min |
| | 2. Pullman: upper heat 220° C./lower heat |
| | 210° C.: 35 min |

The baked bread loaves were stored in a sealed container for one day at a temperature of 20° C. and a humidity of 30%. Then, the specific volume, color difference, hardness, adhesiveness, cohesiveness, fragility, elasticity, chewiness, saccharide composition, and total saccharide content of the bread loaves were determined. Also, the bread loaves were evaluated by sensory testing (taste) (n=6).

(1) Specific Volume

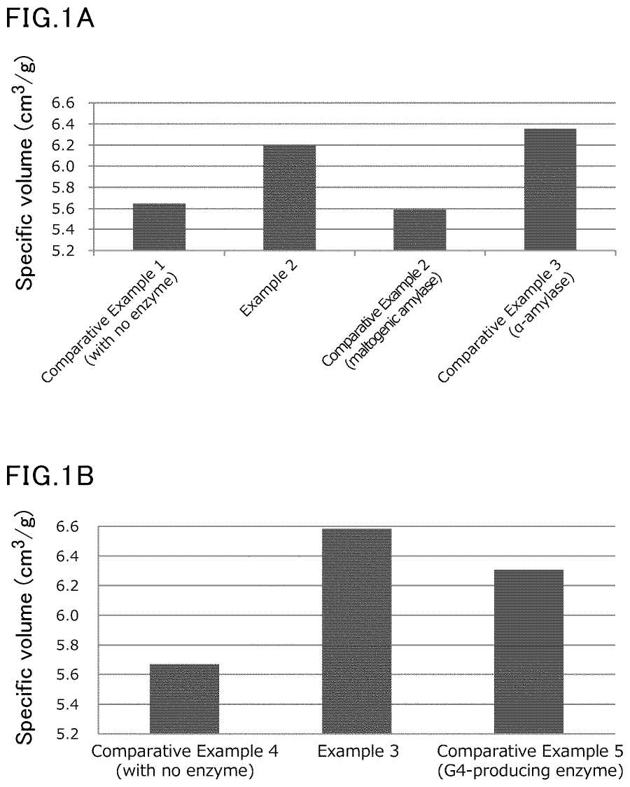

The specific volume means the volume occupied by a unit mass of material. The volume (cm³) and weight (g) of each bread loaf were measured using a laser volume meter to calculate the specific volume (cm³/g). Moreover, since four round top bread loaves were obtained from one dough, their average value was calculated. The laser volume meter used was 3D Laser Volume Measurement Selnac-Win VM2100 (available from ASTEX). FIG. 1A shows the results.

The bread loaves of Example 2 had a specific volume that was considerably greater than that of the bread loaves of Comparative Examples 1 and 2 and comparable to the bread loaves of Comparative Example 3. This is believed to be because the exomaltotetraohydrolase acted on the starch in wheat flour to produce maltotetraose, which was then degraded by the enzymes such as amylase present in the ingredient wheat flour to produce oligosaccharides such as maltose, thereby accelerating fermentation of the bakery yeast. An increase in specific volume leads to an increase in volume and prevention of staling.

(2) Color difference Since four round top bread loaves were obtained from one dough, the baked color at the center (one point) of the surface of each bread loaf was measured with a color difference meter, with the bread loaves with no enzyme as a control, and the average of the measured values was used as the color difference. The color difference meter used was Color meter ZE6000 (available from Nippon Denshoku Industries Co., Ltd.). The color difference means a difference in color between two samples (color stimuli) as defined using ΔL*, Δa*, and Δb*, which are the differences in the coordinates L*, a*, and b*, respectively, in the L*a*b* color system. Since a larger value obtained indicates a greater color difference, the color difference of the bread loaves with an enzyme of each example was determined with the control (with no enzyme) taken as 0. Table 4 shows the results.

TABLE 4

| Quality improving composition | Color difference |
|---|---|
| Comparative Example 1 (with no enzyme) | 0 |
| Example 2 | 7.84 |
| Comparative Example 2 (maltogenic | 2.41 |
| amylase) | |
| Comparative Example 3 (α-amylase) | 3.17 |

The color difference of Example 2 was greater than those of Comparative Examples 1 to 3. This is believed to be because the maltose content in the bread loaves of Example 2 is higher than those of Comparative Examples 1 to 3. The baked color of bread is produced as the saccharides in the bread develop a color through a Maillard reaction and a caramelization reaction. The saccharide most likely to undergo a Maillard reaction is fructose, followed by glucose, maltose, lactose, and sucrose, in decreasing order of likelihood. In Example 2, it is believed that the exomaltotetraohydrolase produces maltotetraose, which is then degraded by the amylase present in the ingredients to produce maltose. Since the bread loaves of Example 2 are not largely different from the bread loaves of Comparative Examples 1 to 3 in terms of fructose and glucose contents, whether a fresh baked color is provided or not is considered to depend on maltose content.

(3) Hardness

The hardness means the maximum test force (N) measured when stress is applied using a plunger. The hardness was calculated from the maximum test force (N) determined when stress was applied to the crumb of Pullman bread using a rheometer. Pullman bread was cut into slices with a width of 3 cm, and four 3-cm-square pieces were cut out from the crumb of the slices and then measured, and their average value was used as the hardness (N). The rheometer used was Sun Rheo Meter CR-500DX (available from Sun Scientific Co., Ltd.). When a bread crumb piece is set in the rheometer, force is applied to the bread twice from above, and the output data, including the magnitude of force and the depth to which the object sank are presented on the stress diagram and texture profile. The stress diagram and texture profile obtained by the rheometer were as described in the instruction manual of the Rheo Data Analkyzer (available from Sun Scientific Co., Ltd.).

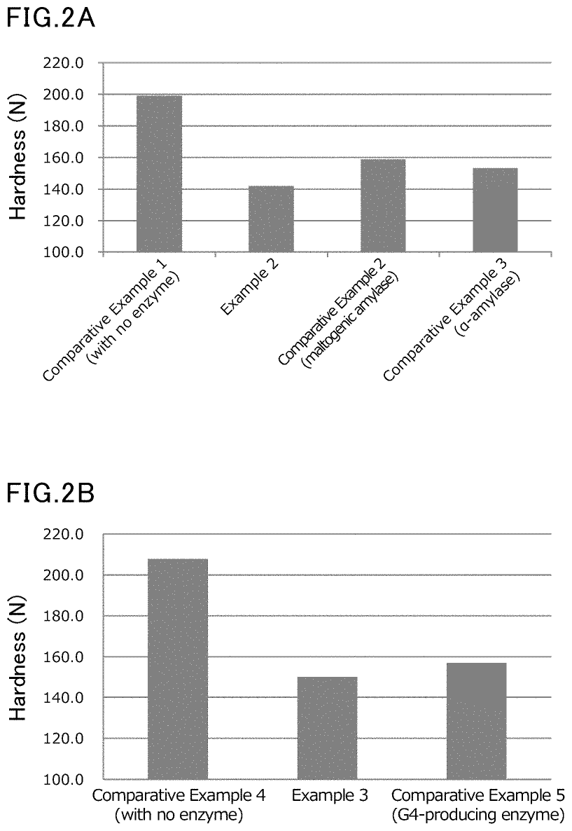

FIG. 2A shows the results.

The bread loaves of Example 2 were considerably softer than the bread loaves of Comparative Example 1 and had a softness equal to or higher than those of the bread loaves of Comparative Examples 2 and 3. This is believed to be because the starch was moderately degraded so that recrystallization of the starch was reduced. Softness (decrease in hardness) of bread leads to prevention of staling.

(4) Adhesiveness

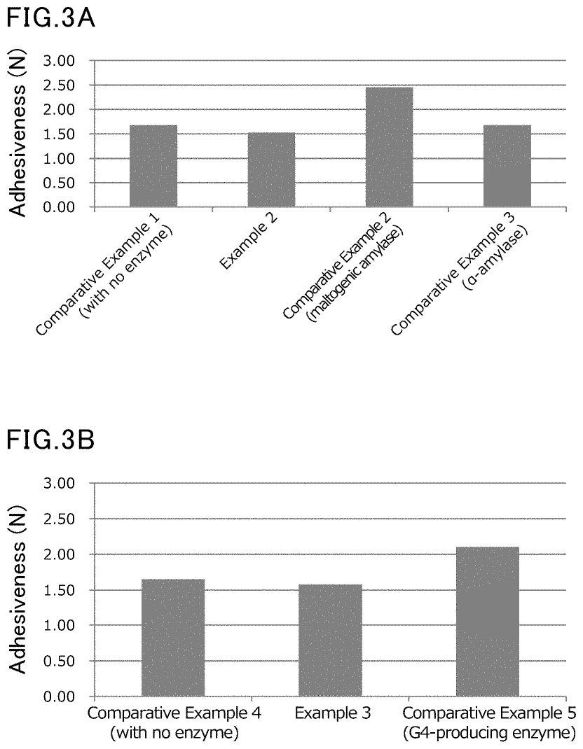

The adhesiveness means the force (N) required to detach the food that adheres to the hand when touched or to the teeth, tongue, or cavity of mouth when eaten. The adhesiveness was determined when stress was applied to the crumb of Pullman bread using a plunger of a rheometer. Pullman bread was cut into slices with a width of 3 cm, and four 3-cm-square pieces were cut out from the crumb of the slices and then measured, and their average value was used as the adhesiveness (N). The rheometer used was Sun Rheo Meter CR-500DX (available from Sun Scientific Co., Ltd.). FIG. 3A shows the results.

The bread loaves of Example 2 had an adhesiveness that was lower than that of the bread loaves of Comparative Examples 2 and 3 and equal to or lower than that of the bread loaves of Comparative Example 1. This is believed to be because the starch was moderately degraded so that excessive gelatinization of the starch was reduced. A decrease in adhesiveness of bread leads to reduction in stickiness (kuchatsuki).

(5) Cohesiveness

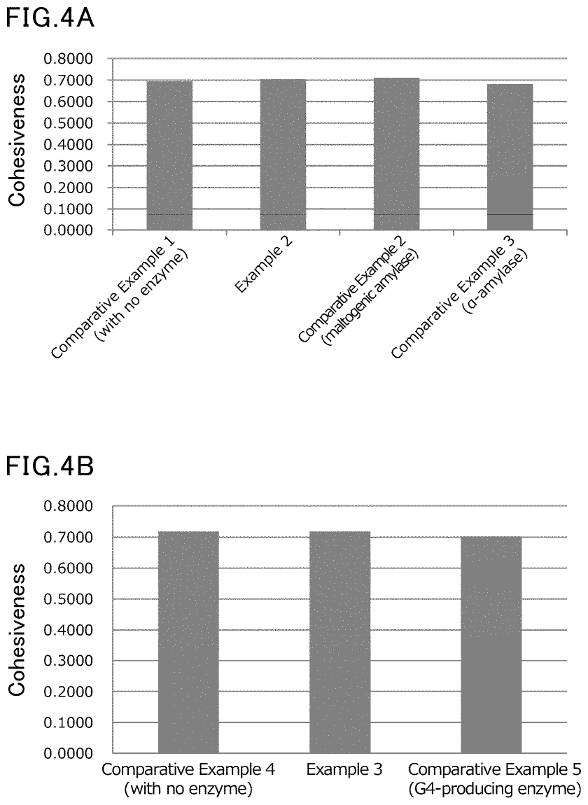

Foods may be deformed or damaged when stress is applied to the foods. The cohesiveness means the ratio between first and second stress areas (energies) when stress is applied to a food twice in a row. The cohesiveness was determined when stress was applied to the crumb of Pullman bread using a plunger of a rheometer. Pullman bread was cut into slices with a width of 3 cm, and four 3-cm-square pieces were cut out from the crumb of the slices and then measured, and their average value was used as the cohesiveness (N). The rheometer used was Sun Rheo Meter CR-500DX (available from Sun Scientific Co., Ltd.). FIG. 4A shows the results.

The bread loaves of Example 2 had substantially the same cohesiveness as the bread loaves of Comparative Examples 1 to 3. It is generally thought that the use of bread quality improving compositions may increase cohesiveness. However, the use of exomaltotetraohydrolase in the bread loaves of Example 2 did not increase cohesiveness. Maintenance of cohesiveness of bread leads to prevention of staling and improvement in melt-in-the-mouth texture of bread.

(6) Fragility

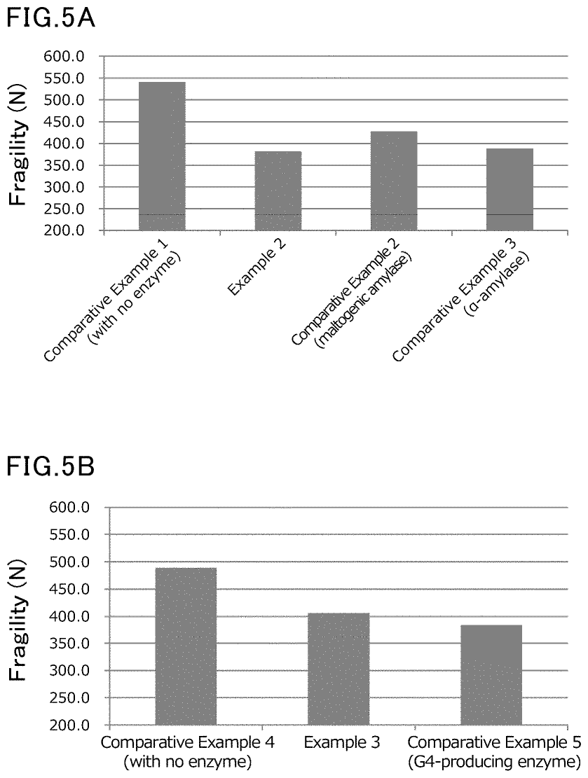

The fragility means the force (N) at which a food breaks down in the mouth. The fragility was determined when stress was applied to the crumb of Pullman bread using a plunger of a rheometer. Pullman bread was cut into slices with a width of 3 cm, and four 3-cm-square pieces were cut out from the crumb of the slices and then measured, and their average value was used as the fragility (N). The rheometer used was Sun Rheo Meter CR-500DX (available from Sun Scientific Co., Ltd.). FIG. 5A shows the results.

The bread loaves of Example 2 were considerably brittler than the bread loaves of Comparative Example 1 and had a fragility equal to or higher than those of Comparative Examples 2 and 3. This is believed to be because the starch was moderately degraded so that recrystallization of the starch was reduced. Fragility of bread leads to prevention of staling and improvement in melt-in-the-mouth texture.

(7) Elasticity

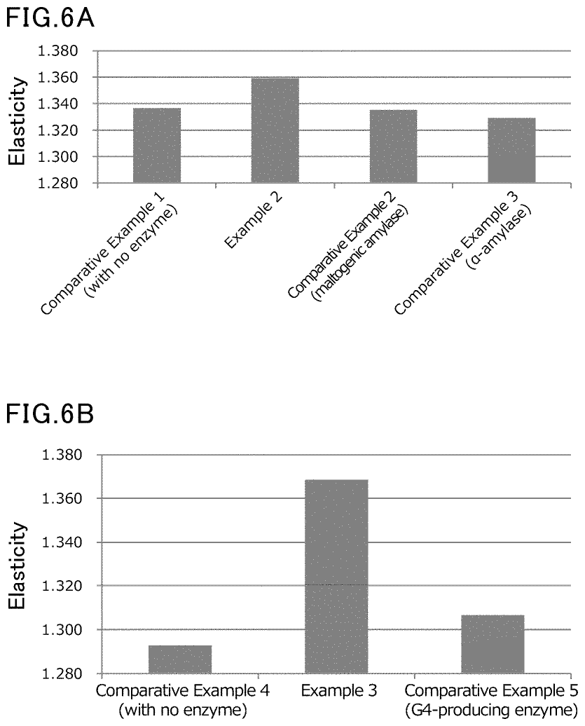

The elasticity means the ratio of a second “indentation displacement” to a first “indentation displacement” when stress is applied to a food twice in a row using a plunger. The elasticity was determined when stress was applied to the crumb of Pullman bread using a plunger of a rheometer. Pullman bread was cut into slices with a width of 3 cm, and four 3-cm-square pieces were cut out from the crumb of the slices and then measured, and their average value was used as the elasticity. The rheometer used was Sun Rheo Meter CR-500DX (available from Sun Scientific Co., Ltd.). FIG. 6A shows the results.

The bread loaves of Example 2 had a higher elasticity than the bread loaves of Comparative Example 1 to 3. This is believed to be because the starch was moderately degraded so that excessive gelatinization was reduced. Elasticity of bread leads to prevention of staling and improvement in springiness.

(8) Chewiness

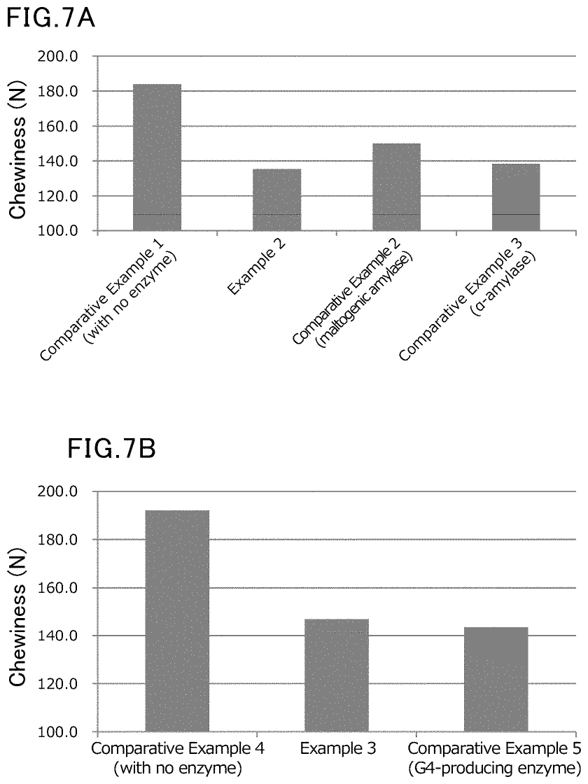

The chewiness means the energy required to chew a solid food until it is ready for swallowing, and is given by the relationship hardness (N)×elasticity×cohesiveness. Pullman bread was cut into slices with a width of 3 cm, and four 3-cm-square pieces were cut out from the crumb of the slices and then measured for hardness, elasticity, and cohesiveness as described above to calculate the chewiness. Further, their average value was determined. The rheometer used was Sun Rheo Meter CR-500DX (available from Sun Scientific Co., Ltd.). FIG. 7A shows the results.

The bread loaves of Example 2 had a chewiness that was lower than that of the bread loaves of Comparative Example 1 and equal to or lower than that of the bread loaves of Comparative Examples 2 and 3. This is believed to be because the starch was moderately degraded so that recrystallization of the starch was reduced. A decrease in chewiness of bread leads to prevention of staling and improvement in melt-in-the-mouth texture.

(9) Saccharide Composition

The saccharide composition was measured through the following processes (i) to (vi).

(i) The bread crumb pieces (four 3-cm cubes after the measurement with a rheometer) are pulverized.

(ii) 5 g of bread powder is weighed into a 50-mL beaker and 30 g of ion-exchange water is added thereto.

(iii) The mixture is stirred at room temperature for 60 minutes (6-barreled stirrer, memory 5, No6 is 6)

(iv) The whole amount of the mixture is placed in a 50-mL centrifuge tube and centrifuged (8000 rpm×10 min).

(v) 2 mL of the supernatant is placed in an Eppendorf tube and centrifuged (14000 rpm×15 min).

(vi) The supernatant is analyzed by HPLC.

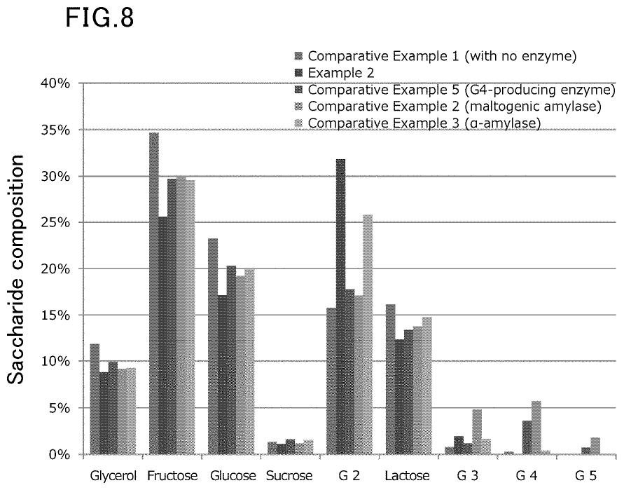

The glycerol, fructose, glucose, sucrose, maltose (G2), lactose, maltotriose (G3), maltotetraose (G4), and maltopentaose (G5) contents were determined based on the HPLC analysis to calculate the percentages (%) of the components. FIG. 8 shows the results.

The bread loaves of Comparative Examples 1 to 3 contained fructose as a main saccharide component. In contrast, the bread loaves of Example 2 contained maltose as a main saccharide component. The above results of the specific volume, color difference, hardness, adhesiveness, cohesiveness, fragility, elasticity, and chewiness of the bread loaves of Example 2 also seem to be due to the presence of maltose as a main saccharide component.

(10) Total Saccharide Content

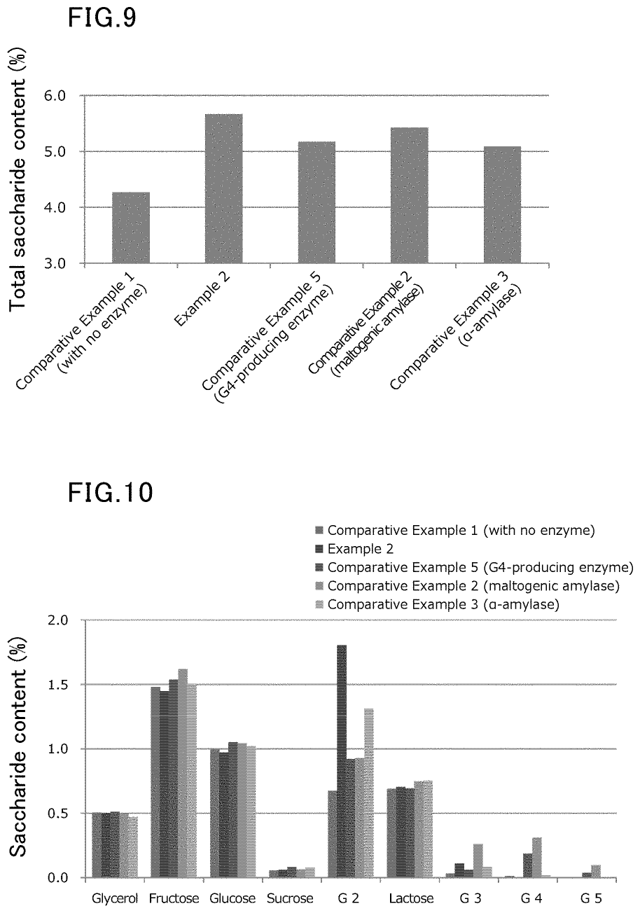

The supernatant obtained in the process (v) in the item (9) was measured by the anthrone-sulfuric acid method. Here, the moisture content in the bread crumb was 40% and the amount of water used in extraction with water was 30 g. FIG. 9 shows the results. In FIG. 9, the vertical axis represents the total saccharide content (unit: %) in the bread crumb. Also, individual saccharide contents were calculated from the saccharide composition and the total saccharide content. FIG. 10 shows the results. In FIG. 10, the vertical axis shows the individual saccharide contents (unit: %) in the bread crumb.

The bread loaves of Example 2 had a higher total saccharide content than the bread loaves of Comparative Examples 1 to 3. Also, the bread loaves of Example 2 were found to have a higher maltose content than the bread loaves of Comparative Examples 1 to 3. These results lead to improvement in baked color, flavor, and food texture.

(11) Sensory Testing

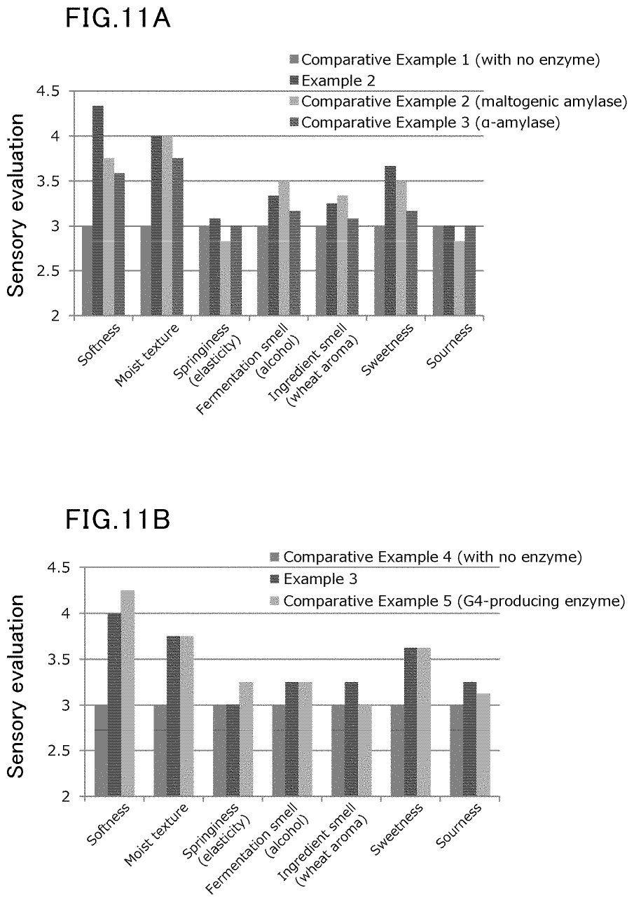

The crumb of the bread loaves on Day 1 after the baking was subjected to sensory testing by six evaluators. The evaluation was made using a 5-point scale from 1 to 5, with the result of the bread loaves with no enzyme set to 3. Here, the “softness” shows whether or not it is easy to chew the bread, with 1 meaning “hard” and 5 meaning “soft”. The “moist texture” shows whether or not the bread has moisture retaining properties when the bread is chewed, with 1 meaning “dry” and 5 meaning “moist”. The “springiness (elasticity)” shows whether or not the bread has elasticity when the bread is chewed, with 1 meaning “crispy” and 5 meaning “springy”. The “fermentation smell (alcohol)” means whether or not the bread has an alcohol smell by itself or when it is chewed, with 1 meaning having no alcohol smell and 5 meaning having an alcohol smell. The “ingredient smell (wheat aroma)” shows whether or not the bread has a wheat smell by itself or when it is chewed, with 1 meaning having a wheat smell and 5 meaning having no wheat smell. The “sweetness” shows whether or not the bread has sweetness, with 1 meaning “not sweet” and 5 meaning “sweet”. The “sourness” shows whether or not the bread has sourness, with 1 meaning having no sourness and 5 meaning having sourness. FIG. 11A shows the results.

The bread loaves of Example 2 scored high in the evaluations of softness, moist texture, and sweetness. It is considered that since the bread loaves of Example 2 contained a large amount of maltose having a flavor different from that of glucose, they had a better flavor than the bread loaves of Comparative Examples 1 to 3. The springiness, fermentation smell, ingredient smell, and sourness of the bread loaves of Example 2 were substantially equal to those of Comparative Examples 1 to 3.

(12) Appearance

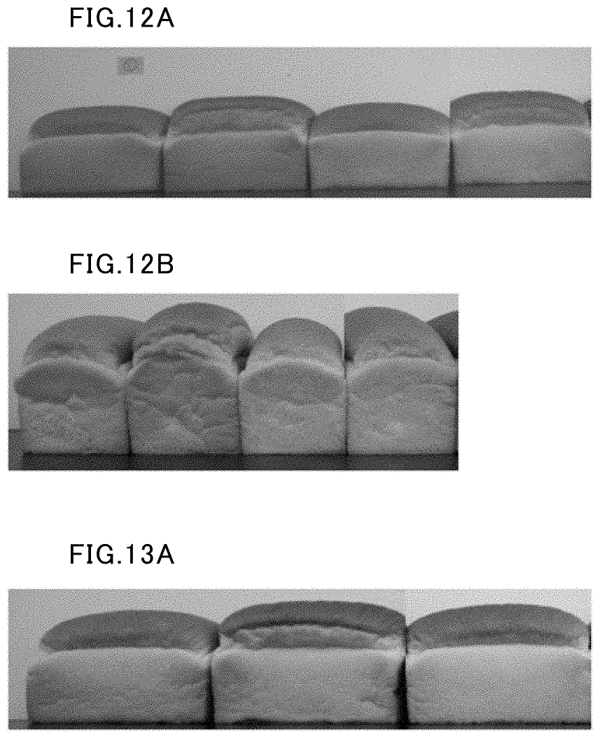

The appearance of the bread loaves are shown in FIG. 12A and FIG. 12B. FIG. 12A and FIG. 12B each show, from the left, the bread loaves of Comparative Example 1 (with no enzyme), Example 2, Comparative Example 2 (maltogenic amylase), and Comparative Example 3 (α-amylase).

The bread loaves of Example 2 had a height that was greater than that of the bread loaves of Comparative Examples 1 and 2 and comparable to the bread loaves of Comparative Example 3. This is believed to be because the exomaltotetraohydrolase acted on the starch in wheat flour to produce oligosaccharides such as maltose, thereby accelerating fermentation of the bakery yeast.

### Example 3 and Comparative Examples 4 and 5

Bread loaves were produced with the bread quality improving composition of Example 1 using a sponge and dough recipe (Example 3). The quality improving composition content was the same as in Example 2. Also, bread loaves were produced with no enzyme (Comparative Example 4) or using a genetically engineered G4-producing enzyme (derived from Pseudomonas saccharophilia, HPLG4, Danisco) (Comparative Example 5) in place of the bread quality improving composition of Example 1. The ingredient contents, the conditions in the sponge step, and the conditions in the final dough step are as shown in Tables 1 to 3. The quality improving composition content was 9.1 ppm for the G4-producing enzyme (Comparative Example 5).

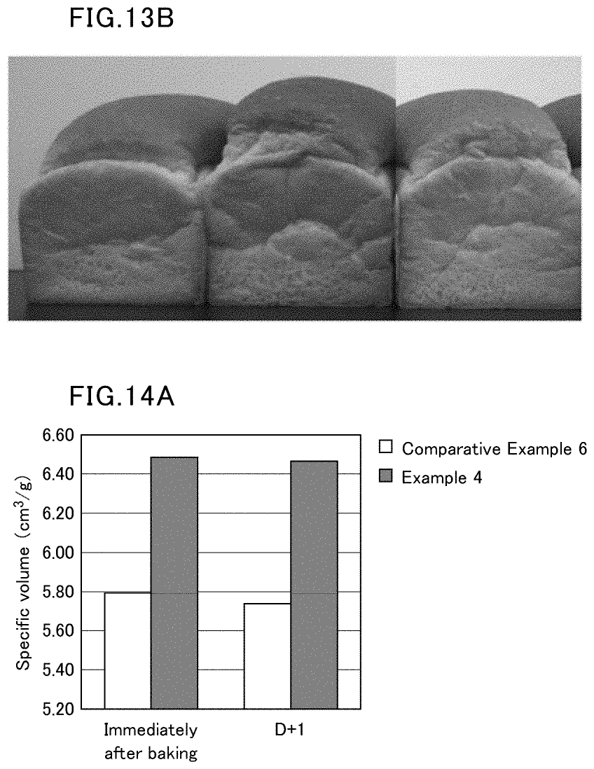

The specific volume (FIG. 1B), color difference (Table 5), hardness (FIG. 2B), adhesiveness (FIG. 3B), cohesiveness (FIG. 4B), fragility (FIG. 5B), elasticity (FIG. 6B), chewiness (FIG. 7B), saccharide composition (FIG. 8), total saccharide content (FIG. 9), and individual saccharide contents (FIG. 10) of the baked bread loaves were measured under the same conditions as in Example 2. Also, sensory testing (n=6) (FIG. 11B), appearance evaluation (FIGS. 13A and 13B), and smell evaluation were performed.

(1) Specific Volume

FIG. 1B shows that the bread loaves of Example 3 had a greater specific volume than the bread loaves of Comparative Examples 4 and 5 and exhibited results similar to the bread loaves of Example 2.

(2) Color Difference

Table 5 shows the color difference measurement results.

TABLE 5

| Quality improving composition | Color difference |
|---|---|
| Comparative Example 4 (with no enzyme) | 0 |
| Example 3 | 9.07 |
| Comparative Example 5 (G4-producing | 7.37 |
| enzyme) | |

Table 5 shows that the bread loaves of Example 3 had a greater color difference than the bread loaves of Comparative Examples 4 and 5 and exhibited results similar to the bread loaves of Example 2.

(3) Hardness

FIG. 2B shows that the bread loaves of Example 3 were softer than the bread loaves of Comparative Examples 4 and 5 and exhibited results similar to the bread loaves of Example 2.

(4) Adhesiveness

FIG. 3B shows that the bread loaves of Example 3 had a lower adhesiveness than the bread loaves of Comparative Examples 4 and 5 and exhibited results similar to the bread loaves of Example 2.

(5) Cohesiveness

FIG. 4B shows that the bread loaves of Example 3 had substantially the same cohesiveness as the bread loaves of Comparative Examples 4 and 5 and exhibited results similar to the bread loaves of Example 2.

(6) Fragility

FIG. 5B shows that the bread loaves of Example 3 were considerably brittler than the bread loaves of Comparative Example 4, had substantially the same cohesiveness as the bread loaves of Comparative Example 5, and exhibited results similar to the bread loaves of Example 2.

(7) Elasticity

FIG. 6B shows that the bread loaves of Example 3 had a higher elasticity than the bread loaves of Comparative Examples 4 and 5 and exhibited results similar to the bread loaves of Example 2.

(8) Chewiness

FIG. 7B shows that the bread loaves of Example 3 had a chewiness that was lower than that of the bread loaves of Comparative Example 4 and equal to that of the bread loaves of Comparative Example 5, and exhibited results similar to the bread loaves of Example 2.

(9) Saccharide Composition

FIG. 8 shows that the bread loaves of Comparative Example 5 contained fructose as a main saccharide component.

(10) Saccharide Content

FIG. 9 shows that the bread loaves of Comparative Example 5 had a total saccharide content equal to or lower than that of the bread loaves of Comparative Examples 2 and 3. FIG. 10 shows that the bread loaves of Comparative Example 5 had a fructose content equal to that of Comparative Examples 2 and 3 and a maltose content equal to or lower than that of Comparative Examples 2 and 3.

(11) Sensory Testing

FIG. 11B shows that the bread loaves of Example 3 scored high in the evaluations of softness, moist texture, and sweetness, and it is considered that they had a flavor that was equal to that of the bread loaves of Comparative Example 5 and better than that of the bread loaves of Comparative Example 4. The springiness, fermentation smell, ingredient smell, and sourness of the bread loaves of Example 3 were substantially equal to those of Comparative Examples 4 and 5.

(12) Appearance

The appearance of the bread loaves is shown in FIG. 13A and FIG. 13B. FIG. 13A and FIG. 13B each show, from the left, the bread loaves of Comparative Example 4 (with no enzyme), Example 3, and Comparative Example 5 (G4-producing enzyme). The bread loaves of Example 3 had a greater height than the bread loaves of Comparative Examples 4 and 5 and exhibited results similar to the bread loaves of Example 2.

(13) Smell

The smell of the bread loaves during baking (n=4) was evaluated by the following procedure. Specifically, the bread dough after completion of the secondary fermentation was placed in a 100-mL screw cap bottle modified to allow us to smell it from the top of the cap. The bottle was placed in an incubator. The bread dough was baked for 30 minutes while the temperature in the incubator was increased from 120° C. to 180° C., and the smell during this process was checked. As a result, Comparative Example 5 had a sweeter, more savory aroma than Comparative Example 4, but Example 3 had a sweet, savory aroma that was stronger than that of Comparative Example 5.

### Example 4 and Comparative Example 6

Variety bread loaves were produced with the bread quality improving composition of Example 1 (Example 4). Also, variety bread loaves were produced with no enzyme (Comparative Example 6) instead of using the bread quality improving composition of Example 1. Tables 6 and 7 show the ingredient contents and the production steps of the variety bread loaves.

TABLE 6

| Ingredient | Example 4 | Comparative Example 6 |
|---|---|---|
| Strong flour | 100 | 100 |
| Quality improving | 200 ppm relative to | — |
| composition | strong flour | |
| US yeast (Oriental | 4 | 4 |
| Yeast Co.,Ltd.) | | |
| Granulated sugar | 15 | 15 |
| Whole egg | 10 | 10 |
| Skim milk powder | 4 | 4 |
| Unsalted butter | 10 | 10 |
| Shortening | 5 | 5 |
| Table salt | 1.5 | 1.5 |
| Water | 50 | 50 |

Each value in Table 6 except for the quality improving composition is expressed in parts by weight based on 100 parts by weight of strong flour. The quality improving composition content in Example 4 was 200 ppm relative to the strong flour.

TABLE 7

| Step | Details of each step |
|---|---|
| Mixing | Add ingredients other than oil and fat (unsalted |
| | butter, shortening) |
| | First speed: 3 min, second speed: 3 min, third |
| | speed: 2 min |
| | Add oil and fat (unsalted butter, shortening) |
| | First speed: 3 min, second speed: 2 min, third |
| | speed: 1 min |
| Final mixing | 27° C. to 28° C. |
| Primary fermentation | 28° C./80%, 30 min |
| Division | Bun: 45 g |
| | Pullman: 210 g × 6 (U-shaped, placed in |
| | opposite directions, × 3) |
| | One-loaf type: 300 g × 4 (moulder, normal |
| | rotation for rolling) |
| Bench rest time | 20 to 30 min |
| Secondary | 35° C./80%, 45 to 60 min |
| fermentation | Pullman (85% of pan) |
| | One-loaf type (1.5 cm above pan) |
| Baking | Upper heat 210° C., lower heat 200° C. |

The variety bread loaves thus produced were stored in a sealed container for one to six days. Thereafter, the specific volume, hardness, saccharide composition, and total saccharide content of the bread loaves were measured. Also, the bread loaves were evaluated by sensory testing (taste).

(1) Specific Volume

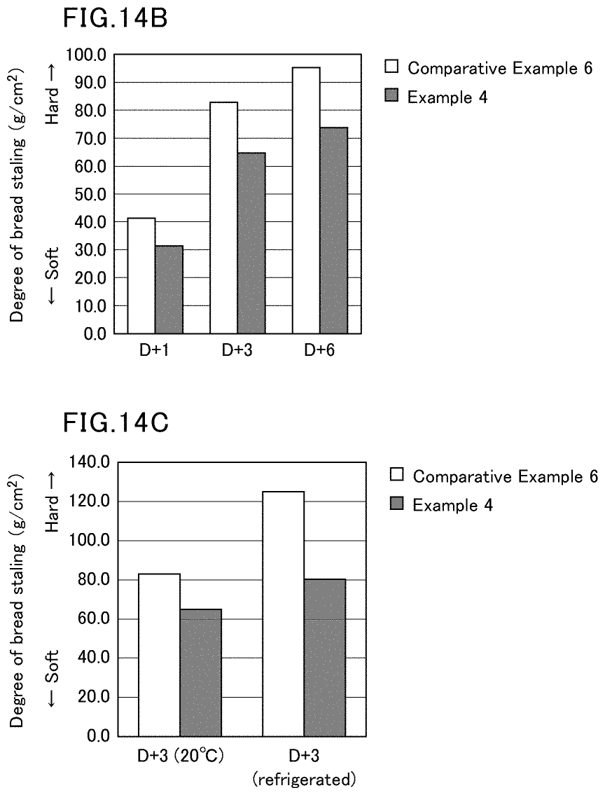

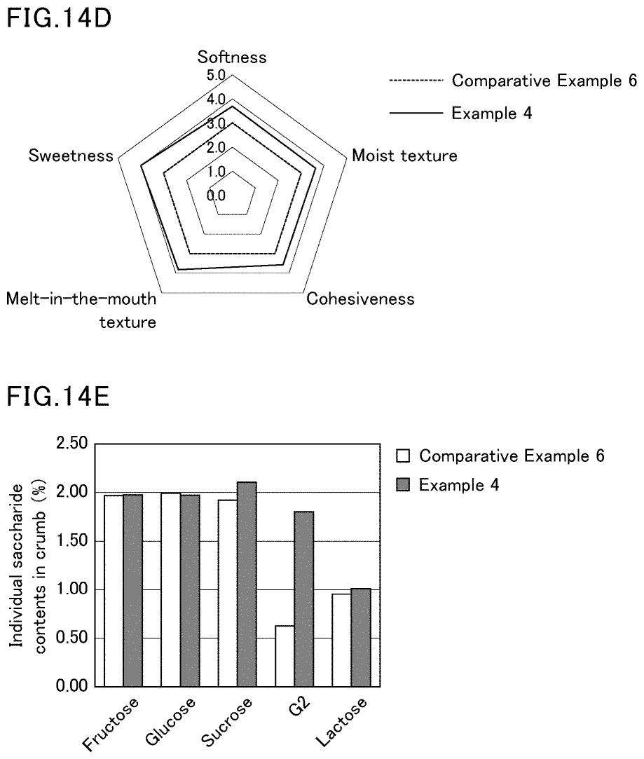

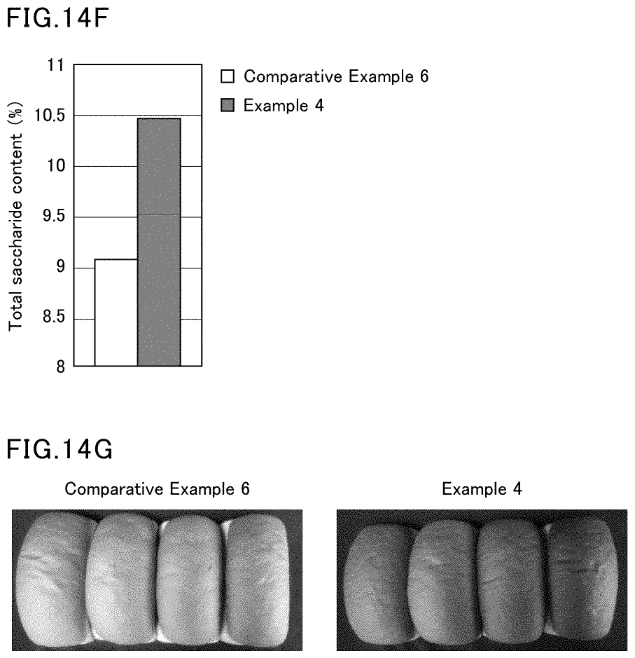

The specific volume of the variety bread loaves immediately after the baking and on Day 1 (stored at a temperature of 20° C. and a humidity of 30%) after the baking was measured as in Example 2. FIG. 14A shows the results. The specific volume of the variety bread loaves of Example 4, both immediately after the baking and on Day 1 after the baking, was increased compared to Comparative Example 6.

(2) Hardness

The hardness of the variety bread loaves on Days 1 to 6 (stored at a temperature of 20° C. and a humidity of 30%) after the baking was measured as in Example 2. FIG. 14B shows the results. On all of Day 1, Day 3, and Day 6 after the baking, the variety bread loaves of Example 4 had a lower hardness (g/cm²) than Comparative Example 6, and they were found to be less likely to stale.

Also, the hardness of the variety bread loaves stored at a temperature of 4° C. for three days after the baking was measured as in Example 2. FIG. 14C shows the results together with the results for storage at 20° C. The variety bread loaves of Comparative Example 6 stored at 4° C. underwent great staling and hardened, while the variety bread loaves of Example 4, even when stored at 4° C., were inhibited from staling. At both storage temperatures, the variety bread loaves of Example 4 had a lower hardness than Comparative Example 6, and they were found to be less likely to stale.

(3) Sensory Testing

The variety bread loaves of Example 4 on Day 1 after the baking were subjected to sensory testing by six evaluators. The evaluation was made using a 5-point scale, with the result of Comparative Example 6 set to 3. Here, the “softness” shows whether or not it is easy to chew the bread, with 1 meaning “hard” and 5 meaning “soft”. The “moist texture” shows whether or not the bread has moisture retaining properties when the bread is chewed, with 1 meaning “dry” and 5 meaning “moist”. The “cohesiveness” shows whether or not the bread, when chewed, is likely to form an aggregate like a dumpling, with 1 meaning high cohesiveness and 5 meaning low cohesiveness. The “melt-in-the-mouth texture” shows whether or not the bread, when chewed, has a melting feeling in the mouth or smoothness, with 1 meaning a poor melt-in-the-mouth texture and 5 meaning a good melt-in-the-mouth texture. The “sweetness” shows whether or not the bread has sweetness, with 1 meaning “not sweet” and 5 meaning “sweet”.

FIG. 14D shows the results. The variety bread loaves of Example 4 had a better taste than Comparative Example 6 in terms of all the items: softness, moist texture, cohesiveness, melt-in-the-mouth texture, and sweetness.

(4) Saccharide Content

The saccharide content was measured as in Example 2. FIG. 14E shows the results. The fructose, glucose, sucrose, and lactose contents of the variety bread loaves of Example 4 were equal to those of Comparative Example 6, while the maltose (G2) content of the variety bread loaves of Example 4 was about three times that of Comparative Example 6.

(5) Total Saccharide Content

The total saccharide content was measured as in Example 2. FIG. 14F shows the results. In FIG. 14F, the vertical axis represents the total saccharide content (%) in the variety bread loaf. The variety bread loaves of Example 4 had a higher total saccharide content than Comparative Example 6.

(6) Baked Color

The appearance of the bread loaves is shown in FIG. 14G. The variety bread loaves of Example 4 had a darker baked color than Comparative Example 6. This is believed to be because the saccharide content increased.

Variety breads contain large amounts of oils, fats, and proteins, and thus the action of enzymes in the breads generally tends to be inhibited easily. However, the quality improver containing exomaltotetraohydrolase also had effects on variety breads, including an increase in specific volume, prevention of staling, improvement in taste and baked color, and an increase in maltose content.

### Example 5 and Comparative Examples 7 to 9

French bread loaves were produced with the bread quality improving composition of Example 1 (Example 5). Also, French bread loaves were produced which contained 0.3 wt % of malt syrup (Comparative Example 7) or 0.6 wt % of malt syrup (Comparative Example 8), or with no enzyme (Comparative Example 9), instead of using the bread quality improving composition of Example 1. Tables 8 and 9 show the ingredient contents and the production steps of the French bread loaves.

TABLE 8

| | | Compar- | Compar- | Compar- |
|---|---|---|---|---|
| | | ative | ative | ative |
| Ingredient | Example 5 | Example 7 | Example 8 | Example 9 |
| Strong flour | 100 | 100 | 100 | 100 |
| Quality | 200 ppm | — | — | — |
| improving | relative to | | | |
| composition | strong flour | | | |
| Bakery yeast | 2 | 2 | 2 | 2 |
| Malt syrup | 0 | 0 | 0.3 | 0.6 |
| Table salt | 2 | 2 | 2 | 2 |
| Water | 68 | 68 | 68 | 68 |

Each value in Table 8 except for the quality improving composition is expressed in parts by weight based on 100 parts by weight of strong flour. The quality improving composition content in Example 5 was 200 ppm relative to the strong flour.

TABLE 9

| Step | Details of each step |
|---|---|
| Mixing | Mix all the ingredients |
| Final mixing | 24° C. |
| Fermentation | 28° C., humidity 80%, 120 min→punching |
| | down→60 min |
| Division | Divide into150 g/piece |
| Bench rest time | Room temperature, 20 min |
| Shaping | Shape into sticks |
| Proofing | 28° C., humidity 80%, 60 min |
| Baking | Upper heat 230° C., lower heat 220° C., 25 min |

The French bread loaves thus produced were stored in a sealed container for one to seven days at a temperature of 20° C. and a humidity of 30%. Thereafter, the specific volume, hardness, and saccharide composition of the bread loaves were measured. Also, the bread loaves were evaluated by sensory testing (taste).

(1) Specific Volume

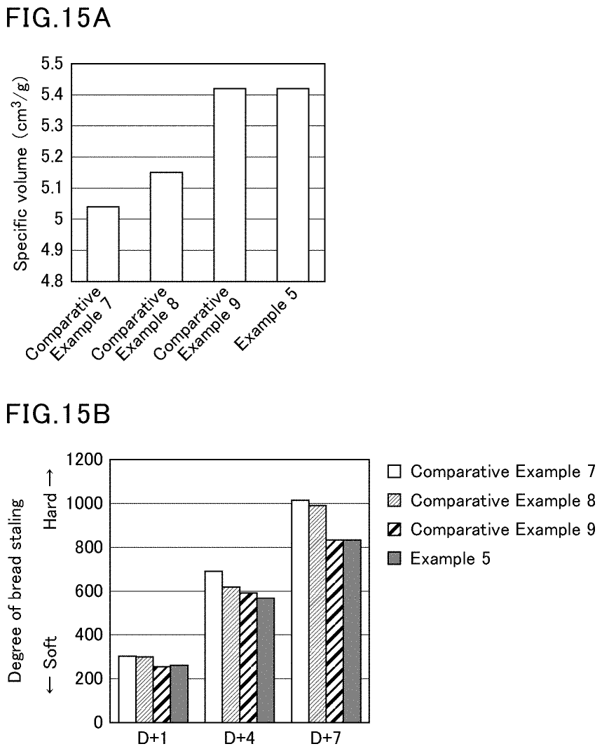

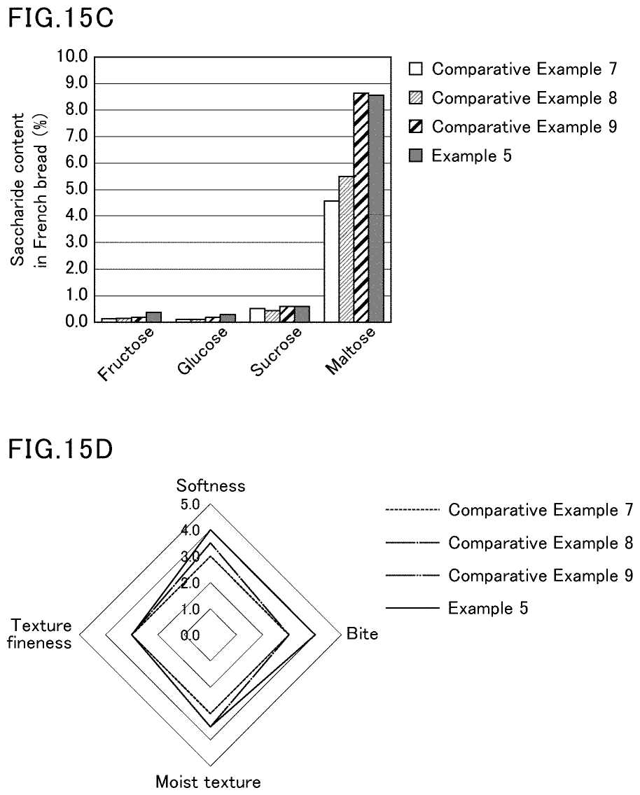

The specific volume of the French bread loaves was measured as in Example 2. FIG. 15A shows the results. The specific volume of the French bread loaves of Example 5 was increased compared to Comparative Example 7. The French bread loaves of Comparative Examples 8 and 9, which contained malt syrup having a volume-increasing effect, had an increased specific volume compared to Comparative Example 7. The French bread loaves of Example 5, although containing no malt syrup, had a specific volume that was greater than that of Comparative Example 8 and equal to that of Comparative Example 9.

(2) Hardness

The hardness of the French bread loaves on Days 1 to 7 after the baking was measured as in Example 2. FIG. 15B shows the results. On all of Day 1, Day 4, and Day 7 after the baking, the French bread loaves of Example 5 had a lower hardness (g/cm²) than Comparative Examples 7 to 9, and they were found to be less likely to stale.

(3) Saccharide Content

The saccharide content was measured as in Example 2. FIG. 15C shows the results. The French bread loaves of Example 5 had a maltose (G2) content that was much higher than that of Comparative Examples 7 to 8 and equal to that of Comparative Example 9 containing 0.6 wt % of malt syrup. Moreover, the French bread loaves of Example 5 had higher fructose and glucose contents than Comparative Examples 7 to 9.

(4) Sensory Testing

The French bread loaves of Example 5 on Day 1 after the baking were subjected to sensory testing by six evaluators. The evaluation was made using a 5-point scale, with the result of Comparative Example 7 set to 3. Here, the “softness” shows whether or not it is easy to chew the bread, with 1 meaning “hard” and 5 meaning “soft”. The “bite” shows whether or not it is easy to bite off the bread, with 1 meaning “not easy to bite” and 5 meaning “easy to bite”. The “moist texture” shows whether or not the bread has moisture retaining properties when the bread is chewed, with 1 meaning “dry” and 5 meaning “moist”. The “texture fineness” shows the visually observed degree of fineness of the texture when the bread is cut, with 1 meaning no fine texture and 5 meaning fine texture. FIG. 15D shows the results. The French bread loaves of Example 5 had a better taste than Comparative Examples 7 and 8 in terms of all the items: softness, bite, moist texture, and texture fineness. The French bread loaves of Example 5 had the same taste as that of Comparative Example 9.

The quality improver containing exomaltotetraohydrolase also had effects on French breads, including an increase in specific volume, prevention of staling, improvement in taste, and an increase in maltose content. Moreover, since French breads only contain wheat flour, salt, yeast, and water as their ingredients, but contain no saccharide, malt syrup is often added to them in order to accelerate fermentation and bring out the taste of the dough. However, the addition of exomaltotetraohydrolase improved the quality of French breads without the need to add malt syrup.

### Example 6 and Comparative Example 10

Croissants were produced with the bread quality improving composition of Example 1 (Example 6). Also, bread loaves were produced with no bread quality improving composition of Example 1, i.e., with no enzyme (Comparative Example 10). Tables 10 and 11 show the ingredient contents and the production steps of the croissants.

TABLE 10

| | | Comparative |
|---|---|---|
| Ingredient | Example 6 | Example 10 |
| LYS D'OR (strong flour, Nisshin | 100 | 100 |
| Seifun Group Inc.) | | |
| Quality improving composition | 200 ppm relative to | — |
| | LYS D'OR | |
| Saf-instant dry yeast (red for low | 2 | 2 |
| sugar dough) | | |
| Granulated sugar | 13 | 13 |
| Unsalted butter | 5 | 5 |
| Whole egg | 5 | 5 |
| Butter sheet | 50 | 50 |
| Table salt | 2.1 | 2.1 |
| Water | 50 | 50 |

Each value in Table 10 except for the quality improving composition is expressed in parts by weight based on 100 parts by weight of strong flour (LYS D′OR). The quality improving composition content in Example 6 was 200 ppm relative to the strong flour.

TABLE 11

| Step | | Details of each step |
|---|---|---|
| Mixing | | Mix Ingredients other than butter sheet. |
| | | Low speed, 3 min → low-mid speed, |
| | | 3 min |
| Final mixing | | 22° C. |
| Fermentation | | Room temperature, 30 min |
| Freezing | | About 4 h, −20° C. |
| Wrapping | Folding | Make a dough sheet |
| butter sheet | operation/ | |
| with dough | sheeter step 1 | |
| | Freezing | About 1 to 2 h |
| | (−20° C.) | |
| | Folding | Make a dough sheet |
| | operation/ | |
| | sheeter step 2 | |

| | |
|---|---|
| Cutting, dividing, | Isosceles triangle |
| shaping of dough | |
| Freezing | Overnight, −20° C. |
| Thawing | Room temperature, about 1 h |
| Proofing | 30° C., 80%, about 3 h |
| Baking | Upper heat 220° C./lower heat 200° C.: |
| | 15 min |

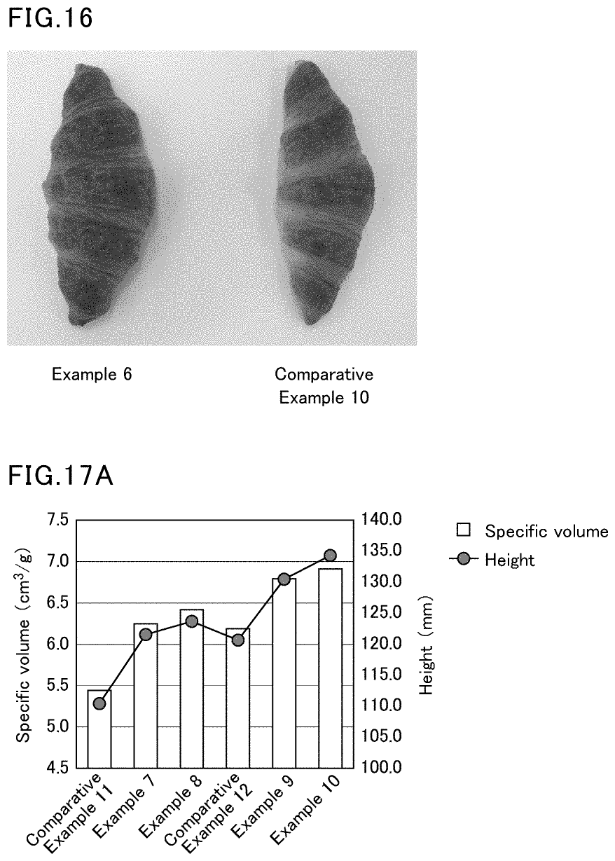

The croissants thus produced were stored in a sealed container for one day at a temperature of 20° C. and a humidity of 30%, and the appearance thereof was observed. FIG. 16 shows the appearance. The croissants of Example 6 had a larger baked size than Comparative Example 10, and had a darker baked color than Comparative Example 10.

Croissants contain large amounts of oils, fats, and proteins, and thus the action of enzymes in the breads generally tends to be inhibited easily. However, the quality improver containing exomaltotetraohydrolase had effects on croissants, including an increase in baked size and improvement in baked color.

### Examples 7 to 10 and Comparative Examples 11 and 12

White bread loaves were produced with the bread quality improving composition of Example 1 by the straight dough method (Examples 7 to 10). Also, white bread loaves were produced with no bread quality improving composition of Example 1 by the straight dough method (Comparative Examples 11 and 12). Tables 12 and 13 show the ingredient contents and the production steps of the white bread loaves.

TABLE 12

| | Comparative | | | Comparative | | | | |
|---|---|---|---|---|---|---|---|---|
| Ingredient | Example 11 | Example 7 | Example 8 | Example 12 | Example 9 | | Example 10 | |
| Strong flour | 100 | 100 | 100 | 100 | 100 | | 100 | |
| Quality | — | 200 ppm | 400 ppm | — | 200 | ppm | 400 | ppm |
| improving | | | | | | | | |
| composition | | | | | | | | |
| Na ascorbate | — | — | — | 30 ppm | 30 | ppm | 30 | ppm |
| (Vitamin C) | | | | | | | | |

| | | | | | | |
|---|---|---|---|---|---|---|
| Fresh yeast | 2 | 2 | 2 | 2 | 2 | 2 |
| Granulated sugar | 6 | 6 | 6 | 6 | 6 | 6 |
| Skim milk powder | 3 | 3 | 3 | 3 | 3 | 3 |
| Shortening | 5 | 5 | 5 | 5 | 5 | 5 |
| Table salt | 2 | 2 | 2 | 2 | 2 | 2 |
| Water | 65 | 65 | 65 | 65 | 65 | 65 |

Each value in Table 12 except for the quality improving composition and Na ascorbate is expressed in parts by weight based on 100 parts by weight of strong flour.

TABLE 13

| Step | Details of each step |
|---|---|
| Mixing | Mix all the Ingredients |
| Final mixing | 27° C. |
| Fermentation | 28° C., humidity 80%, 90 min → punching |
| | down → 30 min |
| Division | One-loaf type: divide into 300 g/piece |
| | Pullman: divide Into 210 g × 3 pieces |
| Bench rest time | Room temperature, 20 min |
| Shaping | One-loaf type: moulder, normal rotation for |
| | rolling |
| | Pullman: U-shaped, placed in opposite directions |
| Proofing | 35° C., humidity 85%, 45 to 90 min |
| Baking | One-loaf type: upper heat 195° C., lower heat |
| | 210° C., 25 min |
| | 1.5-pound Pullman: upper heat 220° C., lower heat |
| | 210° C., 35 min |
| Cooling | Room temperature, 1 h to 1.5 h |

The white bread loaves thus produced were stored in a sealed container at a temperature of 20° C. and a humidity of 30% for one to six days. Thereafter, the specific volume, height, and hardness of the bread loaves were measured. Also, the bread loaves were evaluated by sensory testing (taste).

(1) Specific Volume

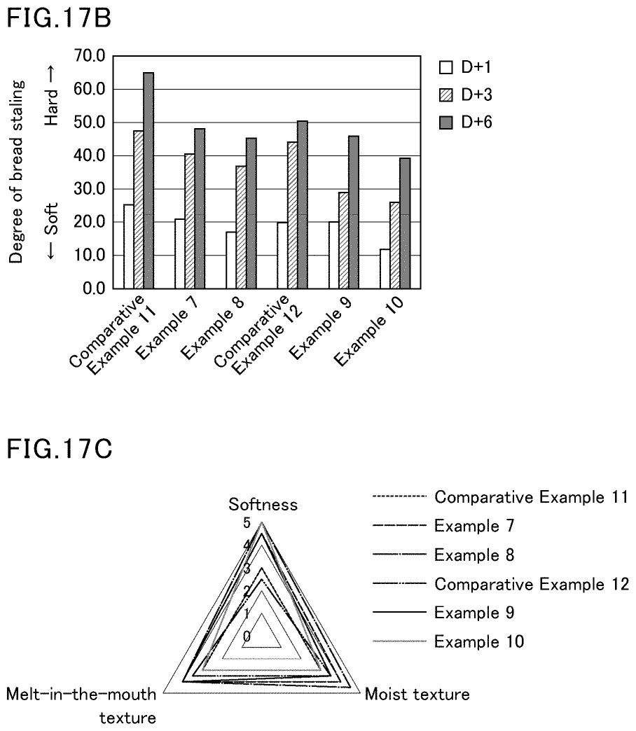

The specific volume of the white bread loaves was measured as in Example 2. Also, the height from the bottom surface to the top of the white bread loaves was measured. FIG. 17A shows the results. The white bread loaves of Examples 7 to 8 had greater specific volume and height than Comparative Example 11. The white bread loaves of Comparative Example 12 containing vitamin C had greater specific volume and height than Comparative Example 11. However, the white bread loaves of Examples 9 and 10 containing vitamin C and the quality improving composition had much greater specific volume and height than Comparative Example 12, and these effects depended on the amount of the quality improving composition added.

(2) Hardness

The hardness of the white bread loaves on Day 1, Day 3, and Day 6 after the baking was measured as in Example 2. FIG. 17B shows the results. On all of Day 1, Day 3, and Day 6 after the baking, the white bread loaves of Examples 7 and 8 had a lower hardness (g/cm²) than Comparative Example 11, and they were found to be less likely to stale. Moreover, the white bread loaves of Comparative Example 12 containing vitamin C showed reduced staling compared to Comparative Example 11, while the white bread loaves of Examples 9 and 10 containing vitamin C and the quality improving composition showed further reduced staling compared to Comparative Example 12, and this effect depended on the amount of the quality improving composition added.

(3) Sensory Testing

The white bread loaves on Day 1 after the baking were subjected to sensory testing by six evaluators. The evaluation was made using a 5-point scale, with the result of Comparative Example 11 set to 3. Here, the “softness” shows whether or not it is easy to chew the bread, with 1 meaning “hard” and 5 meaning “soft”. The “moist texture” shows whether or not the bread has moisture retaining properties when the bread is chewed, with 1 meaning “dry” and 5 meaning “moist”. The “melt-in-the-mouth texture” shows whether or not the bread, when chewed, has a melting feeling in the mouth or smoothness, with 1 meaning a poor melt-in-the-mouth texture and 5 meaning a good melt-in-the-mouth texture. Table 14 and FIG. 17C show the results.

TABLE 14

| | | | Melt-in-the- |
|---|---|---|---|
| | Softness | Moist texture | mouth texture |
| Comparative | 3 | 3 | 3 |
| Example 11 | | | |
| Example 7 | 4.5 | 4 | 4 |
| Example 8 | 5 | 4.5 | 4 |
| Comparative | 2.5 | 3.5 | 3.5 |
| Example 12 | | | |
| Example 9 | 4.5 | 3.5 | 4 |
| Example 10 | 5 | 3 | 3 |

The white bread loaves of Examples 7 and 8 had a better taste than Comparative Example 11 in terms of all the items: softness, moist texture, and melt-in-the-mouth texture, and these effects depended on the amount of the quality improving composition added. Moreover, the white bread loaves of Examples 9 and 10, when containing vitamin C, also had a better taste than Comparative Example 12.

### Examples 11 to 14 and Comparative Examples 13 to 14

White bread loaves were produced with the bread quality improving composition of Example 1 by the sponge and dough method (Examples 11 to 14). Also, white bread loaves were produced with no bread quality improving composition of Example 1 by the sponge and dough method (Comparative Examples 13 to 14). Tables 15 and 16 show the ingredient contents and the production steps of the white bread loaves.

TABLE 15

| | | Comparative | Comparative | | | | |
|---|---|---|---|---|---|---|---|
| Step | Ingredient | Example 13 | Example 14 | Example 11 | Example 12 | Example 13 | Example 14 |
| Sponge | Strong flour | 70 | 70 | 70 | 70 | 70 | 70 |
| | Quality | — | — | 25 ppm | 50 ppm | 100 ppm | 200 ppm |
| | improving | | | | | | |
| | composition | | | | | | |
| | Fresh yeast | 2.5 | 2.5 | 2.5 | 2.5 | 2.5 | 2.5 |
| | Na ascorbate | — | 20, 30, or 40 ppm | | | | |
| | (vitamin C, | | | | | | |
| | as 1% powder) | | | | | | |
| | Water | 40 | 40 | 40 | 40 | 40 | 40 |
| Final | Sponge | Whole | Whole | Whole | Whole | Whole | Whole |
| dough | | amount | amount | amount | amount | amount | amount |
| | Strong flour | 30 | 30 | 30 | 30 | 30 | 30 |
| | Granulated | 6 | 6 | 6 | 6 | 6 | 6 |
| | sugar | | | | | | |
| | Skim milk | 3 | 3 | 3 | 3 | 3 | 3 |
| | powder | | | | | | |
| | Shortening | 5 | 5 | 5 | 5 | 5 | 5 |
| | Table salt | 2 | 2 | 2 | 2 | 2 | 2 |
| | Water | 28 | 28 | 28 | 28 | 28 | 28 |

Each value in Table 15 except for the quality improving composition and Na ascorbate is expressed in parts by weight based on 100 parts by weight of the combined amount of the strong flour used in the sponge and the strong flour added to the final dough.

TABLE 16

| Step | Details of each step |
|---|---|
| Sponge mixing | Final dough temperature: 24° C. |
| Sponge fermentation | 28° C., humidity 80%, 4 h |
| Final dough mixing | Final dough temperature: 27° C. |
| Floor time | 28° C., 15 min |
| Division | One-loaf type: divide into 300 g × 4 pieces |
| | Pullman: divide into 210 g × 6 pieces |
| Bench rest time | Room temperature, 15 min |
| Shaping | Shaping |
| Proofing | 35° C., humidity 85%, 45 to 60 min |
| Baking | One-loaf type: upper heat 195° C., lower |
| | heat 210° C., 25 min |
| | 1.5-pound Pullman: upper heat 220° C., lower |
| | heat 210° C., 35 min |
| Cooling | Room temperature, 1 h to 1.5 h |

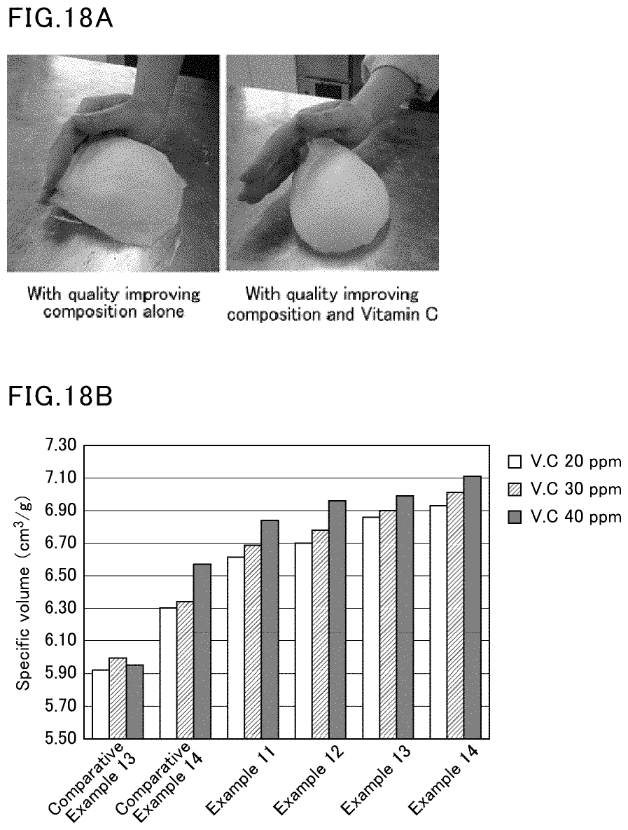

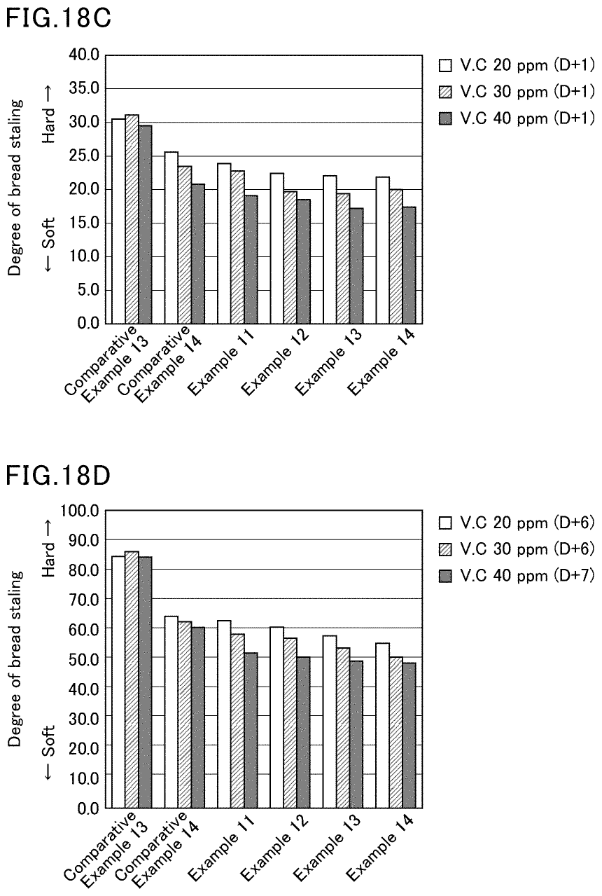

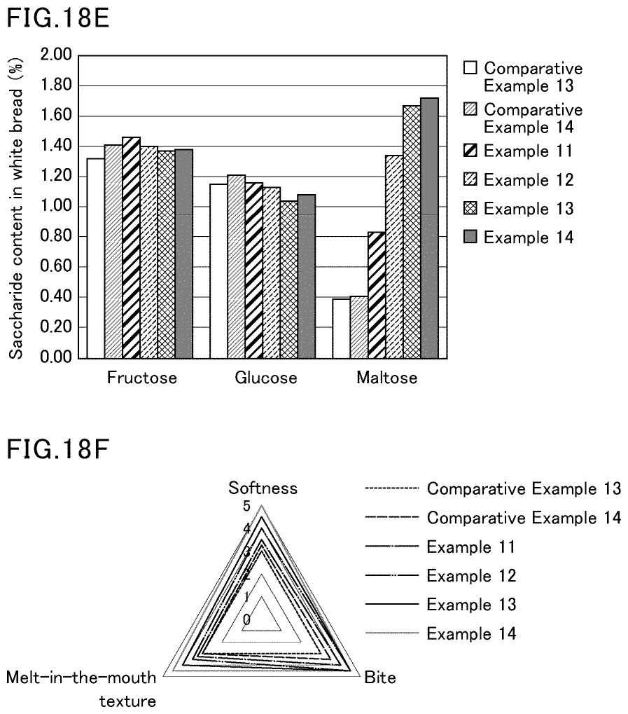

FIG. 18A shows the appearance of the bread dough in the step of shaping after dough division in the sponge and dough method. The combination of the quality improving composition and vitamin C reduced the stickiness of the bread dough. This effect was observed not only on the dough for white bread prepared in the sponge and dough method but also on the bread dough prepared by other methods.

The white bread loaves thus produced were stored in a sealed container at a temperature of 20° C. and a humidity of 30% for one to seven days. Thereafter, the specific volume, hardness, and saccharide content of the bread loaves were measured. Also, the bread loaves were evaluated by sensory testing (taste).

(1) Specific Volume

The specific volume of the white bread loaves was measured as in Example 2. FIG. 18B shows the results. The white bread loaves of Comparative Example 14 containing vitamin C had a greater specific volume than Comparative Example 13. The white bread loaves of Examples 11 to 14 combining the quality improving composition with vitamin C had an even greater specific volume, and this increasing effect was dependent on the amount of the quality improving composition added.

(2) Hardness

The hardness of the white bread loaves on Day 1 after the baking was measured as in Example 2. FIG. 18C shows the results. The white bread loaves of Comparative Example 14 containing vitamin C had a lower hardness (g/cm²) than Comparative Example 13, and they were found to be less likely to stale. The white bread loaves of Examples 11 to 14 combining the quality improving composition with vitamin C showed further reduced staling, and this effect was dependent on the amount of the quality improving composition added.

Also, the hardness of the white bread loaves on Day 6 or 7 after the baking was measured as in Example 2. FIG. 18D shows the results. The anti-staling effect on bread caused by the combination of the quality improving composition and vitamin C was maintained also on Days 6 to 7 after the baking.

(3) Saccharide Content

The saccharide content of the white bread loaves on Day 1 after the baking was measured as in Example 2. FIG. 18E shows the results. The presence of the vitamin did not affect the saccharide composition of the white bread loaves of Comparative Examples 13 to 14. The maltose content of the white bread loaves of Examples 11 to 14 containing was greatly increased depending on the quality improving composition content.

(4) Sensory Testing

The white bread loaves on Day 1 after the baking were subjected to sensory testing by six evaluators. The bread loaves of Comparative Example 14 and Examples 11 to 14 contained 40 ppm sodium ascorbate. The evaluation was made using a 5-point scale, with the result of Comparative Example 13 set to 3. Here, the “softness” shows whether or not it is easy to chew the bread, with 1 meaning “hard” and 5 meaning “soft”. The “bite” shows whether or not it is easy to bite off the bread, with 1 meaning “not easy to bite” and 5 meaning “easy to bite”. The “melt-in-the-mouth texture” shows whether or not the bread, when chewed, has a melting feeling in the mouth or smoothness, with 1 meaning a poor melt-in-the-mouth texture and 5 meaning a good melt-in-the-mouth texture. Table 17 and FIG. 18F show the results.

TABLE 17

| | | | Melt-in-the- |
|---|---|---|---|
| | Softness | Bite | mouth texture |
| Comparative | 3.0 | 3.0 | 3.0 |
| Example 13 | | | |
| Comparative | 3.3 | 3.5 | 3.0 |
| Example 14 | | | |
| Example 11 | 3.5 | 4.0 | 3.3 |
| Example 12 | 4.0 | 4.5 | 3.5 |
| Example 13 | 4.5 | 4.5 | 4.0 |
| Example 14 | 5.0 | 4.5 | 4.5 |

The white bread loaves of Comparative Example 14 containing vitamin C had a food texture equal to or higher than that of Comparative Example 13, while the softness, bite, and melt-in-the-mouth texture of Examples 11 to 14 combining the quality improving composition with vitamin C were all greatly improved compared to Comparative Examples 13 and 14, and these effects were dependent on the amount of the quality improving composition added.

<Comprehensive Evaluation of Exomaltotetraohydrolase>

The exomaltotetraohydrolase significantly improved the baked color, food texture (stickiness (kuchatsuki), springiness, melt-in-the-mouth texture), and flavor of bread as compared with the maltogenic amylase and α-amylase. Moreover, the exomaltotetraohydrolase, although being a non-genetically engineered product, improved the baked color, food texture, and flavor of bread as compared with the genetically engineered G4-producing enzyme. The exomaltotetraohydrolase was better than the conventional quality improving compositions also in terms of increase in volume, prevention of staling, and acceleration of fermentation.

The effects of the bread quality improving composition on bread were comprehensively evaluated based on the above evaluation results. Table 18 shows the relationships between the evaluation items for the examples and the comprehensive evaluation items.

TABLE 18

| Comprehensive evaluation item | Evaluation item for examples |
|---|---|
| Increase in volume | Specific volume |
| Improvement in baked color | Color difference |
| prevention of staling | Specific volume, hardness, cohesive- |
| | ness, fragility, elasticity, chewi- |
| | ness, sensory evaluation |
| Reduction in stickiness | Adhesiveness, sensory evaluation |
| (kuchatsuki) | |
| Improvement in springiness | Elasticity, sensory evaluation |
| Improvement in melt-in- | Chewiness, cohesiveness, fragility, |
| the-mouth texture | sensory evaluation |
| Improvement in flavor | Sensory evaluation, smell evaluation |
| Acceleration of fermentation | Specific volume, smell evaluation |

The comprehensive evaluation was made based on the following criteria.
A: A significant effect was observed compared to the control with no enzyme.
B: An effect was observed compared to the control with no enzyme.
C: A slight effect was observed compared to the control with no enzyme.
D: No or poor effect was observed compared to the control with no enzyme.

Table 19 shows the comprehensive evaluation results.

TABLE 19

| | | | | | | Improvement | | |
|---|---|---|---|---|---|---|---|---|
| | | | Preven- | Reduction in | Improve- | in melt-in- | Improve- | Acceler- |
| | Increase | Improvement | tion of | stickiness | ment in | the-mouth | ment in | ation of |
| Quality improving composition | in volume | in baked color | staling | (kuchatsuki) | springiness | texture | flavor | fermentation |
| Exomaltotetraohydrolase | A | A | B | B | A | A | A | A |
| (Examples 1, 3 to 14) | | | | | | | | |
| G4-producing enzyme | B | B | B | D | B | A | B | B |
| (Comparative Example 5) | | | | | | | | |
| Maltogenic amylase | D | C | B | D | B | B | B | C |
| (Comparative Example 2) | | | | | | | | |
| α-Amylase | A | C | B | B | B | C | B | B |
| (Comparative Example 3) | | | | | | | | |
| With no enzyme | D | D | D | B | D | D | D | D |
| (Comparative Examples | | | | | | | | |
| 1, 4 to 14) | | | | | | | | |

1. A bread quality improver, comprising exomaltotetraohydrolase.

2. The quality improver according to claim 1,
wherein the quality improver is for improving baked color.

3. The quality improver according to claim 1,
wherein the quality improver is for improving food texture.

4. The quality improver according to claim 1,
wherein the quality improver is for improving flavor.

5. The quality improver according to claim 1, which is designed to cause production of saccharides mainly including maltose in bread dough.

6. The quality improver according to claim 1,
wherein the saccharides mainly including maltose are produced by degradation by amylase of maltotetraose that is produced by the action of the exomaltotetraohydrolase.

7. The quality improver according to claim 1,
wherein the exomaltotetraohydrolase is derived from Pseudomonas stutzeri.

8. A bread quality improving composition, comprising the quality improver according to claim 1.

9. A method of producing bread, comprising adding the composition according to claim 8 to at least one bread dough ingredient to increase maltose content.

10. Bread, produced by the method according to claim 9.

11. Bread, comprising saccharides mainly including maltose.
